(19) United States
(12) Patent Application Publication
(10) Pub. No.: US 2006/0019994 A1
(43) Pub. Date: Jan. 26, 2006

(54) I-sulphonlyl piperidine derivatives

(76) Inventors: Jeremy Nicholas Burrows, Macclesfield (GB); Howard Tucker, Macclesfield (GB)

Correspondence Address: FISH & RICHARDSON P.C., P.O BOX 1022, MINNEAPOLIS, MN, 55440-1022, US

(21) Appl. No.: 10/527,209

(22) PCT Filed: Sep. 9, 2003
(86) PCT No.: PCT/GB03/03937

§ 371 Date: Mar. 10, 2005

(30) Foreign Application Priority Data
Sep. 13, 2002 (GB) 0221250.4

(51) Int. Cl. A61K 31/454; C07D 417/02; C07D 413/02

(52) U.S. Cl. 514/326; 546/208; 546/209

(57) ABSTRACT

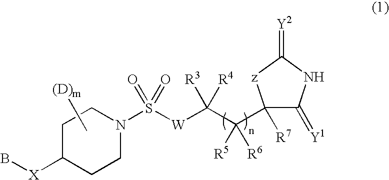

Piperidine derivatives of formula (1) that are useful in the inhibition of metalloproteinases, in particular TNF-α Converting Enzyme (TACE) and thus in the treatment of autoimmune disease, allergic/atopic diseases, transplant rejection, graft versus host disease, cardiovascular disease, reperfusion injury and malignancy.

The present invention relates to compounds useful in the inhibition of metalloproteinases and in particular to pharmaceutical compositions comprising these, as well as their use.

The compounds of this invention are inhibitors of one or more metalloproteinase enzymes and are particularly effective as inhibitors of TNF-α (Tumour Necrosis Factor-α) Production. Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMP) such as the collagenases (MMP1, MMP8, MMP13), the gelatinases (MMP2, MMP9), the stromelysins (MMP3, MMP10, MMP11), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF-α converting enzymes (ADAM10 and TACE); the ADAM-TS family (for example ADAM-TS1 and ADAM-TS4); the astacin family which include enzymes such as procollagen processing proteinase (PCP); and other metalloproteinases such as the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biologically important cell mediators, such as tumour necrosis factor-α (TNF-α); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many disease conditions. Inhibition of the activity of one or more metalloproteinases may well be of benefit in these disease conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema and dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; and extracellular matrix remodelling observed in cardiovascular diseases such as restenosis and atheroscelerosis.

A number of metalloproteinase inhibitors are known; different classes of compounds may have different degrees of potency and selectivity for inhibiting various metalloproteinases. We have discovered a class of compounds that are inhibitors of metalloproteinases and are of particular interest in inhibiting TACE. The compounds of this invention have beneficial potency and/or pharmacokinetic properties.

TACE (also known as ADAM17) which has been isolated and cloned [R. A. Black et al. (1997) Nature 385:729-733; M. L. Moss et al. (1997) Nature 385:733-736] is a member of the admalysin family of metalloproteins. TACE has been shown to be responsible for the cleavage of pro-TNF-α, a 26 kDa membrane bound protein to release 17 kDa biologically active soluble TNF-α. [Schlondorff et al. (2000) Biochem. J. 347: 131-138]. TACE mRNA is found in most tissues, however TNF-α is produced primarily by activated monocytes, macrophages and T lymphocytes. TNF-α has been implicated in a wide range of pro-inflammatory biological processes including induction of adhesion molecules and chemokines to promote cell trafficking, induction of matrix destroying enzymes, activation of fibroblasts to produce prostaglandins and activation of the immune system [Aggarwal et al (1996) Eur. Cytokine Netw. 7: 93-124]. Clinical use of the anti-TNF biologicals has shown TNF-α to play an important role in a range of inflammatory diseases including rheumatoid arthritis, Crohn's disease and psoriasis [Onrust et al (1998) Biodrugs 10: 397-422, Jarvis et al (1999) Drugs 57:945-964]. TACE activity has also been implicated in the shedding of other membrane bound proteins including TGFα, p75 & p55 TNF receptors, L-selectin and amyloid precursor protein [Black (2002) Int. J. Biochem. Cell Biol. 34: 1-5]. The biology of TACE inhibition has recently been reviewed and shows TACE to have a central role in TNF-α production and selective TACE inhibitors to have equal, and possibly greater, efficacy in the collagen induced arthritis model of RA than strategies that directly neutralise TNF-α [Newton et al (2001) Ann. Rheum. Dis. 60: iii25-iii32].

A TACE inhibitor might therefore be expected to show efficacy in all disease where TNF-α has been implicated including, but not limited to, inflammatory diseases including rheumatoid arthritis and psoriasis, autoimmune diseases, allergic/atopic diseases, transplant rejection and graft versus host disease, cardiovascular disease, reperfusion injury, malignancy and other proliferative diseases. A TACE inhibitor might also be effective against respiratory disease such as asthma and chronic obstructive pulmonary diseases (referred to herein as COPD).

Metalloproteinase inhibitors are known in the art. WO 02/074750 and WO 02/074767 disclose compounds comprising a metal binding group that are inhibitors of metalloproteinases. WO 02/074751 also disclosed compounds that are inhibitors of metalloproteinases and especially MMP12.

We are able to provide further compounds that have metalloproteinase inhibitory activity, and are in particular inhibitors of TACE (ADAM17).

According to the first aspect of the present invention there is provided a compound of formula (1), a pharmaceutically acceptable salt or in vivo hydrolysable ester thereof: 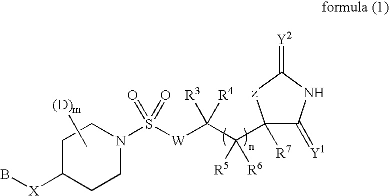
wherein:
Y¹ and Y² are independently O or S;
z is NR⁸, O or S;
n is 0 or 1;
W is NR¹, CR¹R² or a bond;
m is 0 or 1;
D is hydrogen, C₁₋₄alkyl, C₃₋₆cycloalkyl or fluoro;
X is —(CR¹²R¹³)ₜ-Q-(CR¹⁴R¹⁵)ᵤ— where t and u are independently 0 or 1 and Q is O, S, SO or SO₂;
B is a group selected from aryl, heteroaryl and heterocyclyl, where each group is optionally substituted by one or more groups independently selected from nitro, trifluoromethyl, trifluoromethoxy, halo, cyano, C₁₋₄alkyl (optionally substituted by R⁹ or C₁₋₄alkoxy or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy; with the provisos that: when n is 1 and W is NR¹, CR¹R² or a bond; or when n is 0 and W is CR¹R²; then B is a group selected from aryl, heteroaryl and heterocyclyl, where each group is optionally substituted by one or more groups independently selected from nitro, trifluoromethyl, trifluoromethoxy, halo, cyano, C₁₋₄alkyl (optionally substituted by R⁹ or C₁₋₄alkoxy or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy; and when n is 0 and W is NR¹ or a bond; then B is a group selected from bicyclic aryl, bicyclic heteroaryl and bicyclic heterocyclyl, where each group is optionally substituted by one or more groups independently selected from nitro, trifluoromethyl, trifluoromethoxy, halo, cyano, C₁₋₄alkyl (optionally substituted by R⁹ or C₁₋₄alkoxy or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy; R¹ and R² are independently hydrogen or a group selected from C₁₋₆alkyl, C₂₋₆alkenyl, C₂₋₆alkynyl, C₃₋₆cycloalkyl and C₅₋₆cycloalkenyl which group may be optionally substituted by halo, cyano, hydroxy or C₁₋₄alkoxy; R³, R⁴, R⁵ and R⁶ are independently hydrogen or a group selected from C₁₋₆alkyl, C₂₋₆alkenyl, C₂₋₆alkynyl, C₃₋₆cycloalkyl, C₅₋₆cycloalkenyl, aryl, heteroaryl and heterocyclyl which group is optionally substituted by one or more substituents independently selected from halo, nitro, cyano, trifluoromethyl, trifluoromethyloxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, —OR¹⁸, —SR¹⁹, —SOR¹⁹, —SO₂R¹⁹, —COR¹⁹, —CO₂R¹⁸, —CONR¹⁸R²⁰, —NR¹⁶COR¹⁸, —SO₂NR¹⁸R²⁰ and —NR¹⁶SO₂R¹⁹; or R¹ and R³ together with the nitrogen or carbon atoms and carbon atom to which they are respectively attached form a saturated 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl; or R³ and R⁴ together form a saturated 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl; or R⁵ and R⁶ together form a saturated 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl; R⁷ is hydrogen or a group selected from C₁₋₆alkyl, C₂₋₆alkenyl, C₂₋₆alkynyl, heteroalkyl, C₃₋₇Cycloalkyl, aryl, heteroaryl or heterocyclyl where the group is optionally substituted by halo, C₁₋₄alkyl, C₁₋₄alkoxy, C₃₋₇cycloalkyl, heterocyclyl, aryl, heteroaryl or heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, —COC₁₋₄alkyl, —OR²¹, —CO₂R²¹, —SR²⁵, —SOR²⁵, —SO₂R²⁵, —NR²¹COR²², —CONR²¹R²² and —NHCONR²¹R²²; or R³ and R⁷ together with the carbon atoms to which they are each attached and (CR⁵R⁶)ₙ form a saturated 5- to 7-membered ring optionally containing a heteroatom group selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl; R⁸ is selected from hydrogen, C₁₋₆alkyl and haloC₁₋₆alkyl; R⁹ and R¹⁰ are independently hydrogen, C₁₋₆alkyl or C₃₋₆cycloalkyl; or R⁹ and R¹⁰ together with the nitrogen to which they are attached form a heterocyclic 4 to 7-membered ring. R¹¹ is C₁₋₆alkyl or C₃₋₆cycloalkyl; R¹², R¹³, R¹⁴ and R¹⁵ are independently selected from hydrogen, C₁₋₆alkyl and C₃₋₆cycloalkyl; R¹⁶ is hydrogen or C₁₋₆alkyl; R¹⁷ is selected from halo, C₁₋₆alkyl, C₃₋₆cycloalkyl and C₁₋₆alkoxy; R¹⁸ is hydrogen or a group selected from C₁₋₆alkyl, C₃₋₆cycloalkyl, C₅₋₇cycloalkenyl, saturated heterocyclyl, aryl, heteroaryl, arylC₁₋₄alkyl and heteroarylC₁₋₄alkyl which group is optionally substituted by one or more halo; R¹⁹ and R²⁵ are independently a group selected from C₁₋₆alkyl, C₃₋₆cycloalkyl, C₅₋₇cycloalkenyl, saturated heterocyclyl, aryl, heteroaryl, arylC₁₋₄alkyl and heteroarylC₁₋₄alkyl which group is optionally substituted by one or more halo; R²⁰ is hydrogen, C₁₋₆alkyl or C₃₋₆cycloalkyl; R¹⁹ and R²⁰ together with the nitrogen to which they are attached form a heterocyclic 4- to 7-membered ring; R²¹ and R²² independently hydrogen, C₁₋₄alkyl, haloC₁₋₄alkyl, aryl and arylC₁₋₄alkyl; or R²¹ and R²² together with the nitrogen to which they are attached form a heterocyclic 5- to 6-membered ring.

According to a second aspect of the invention there is provided a compound of formula (1), a pharmaceutically acceptable salt or in vivo hydrolysable ester thereof wherein:
Y¹ and Y² are independently O or S;
z is NR⁸, O or S;
n is 0;
W is NR¹ or a bond;
m is 0 or 1;
D is hydrogen, C₁₋₄alkyl, C₃₋₆cycloalkyl or fluoro;
X is —(CR¹²R¹³)ₜ-Q-(CR¹⁴R¹⁵)^{u}— where t and u are independently 0 or 1 and Q is O, S, SO or SO₂;
B is a group selected from aryl, heteroaryl and heterocyclyl, where each group is optionally substituted by one or more groups independently selected from nitro, trifluoromethyl, trifluoromethoxy, halo, cyano, C₁₋₄alkyl (optionally substituted by R⁹ or C₁₋₄alkoxy or one or more halo), C₂₋₄alkenyl (optionally substituted by halo or R⁹), C₂₋₄alkynyl (optionally substituted by halo or R⁹), C₃₋₆cycloalkyl (optionally substituted by R⁹ or one or more halo), C₅₋₆cycloalkenyl (optionally substituted by halo or R⁹), aryl (optionally substituted by halo or C₁₋₄alkyl), heteroaryl (optionally substituted by halo or C₁₋₄alkyl), heterocyclyl (optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, OR⁹, —CONR⁹R¹⁰ and —NR⁹COR¹⁰; R¹ is hydrogen or a group selected from C₁₋₆alkyl, C₂₋₆alkenyl, C₂₋₆alkynyl, C₃₋₆cycloalkyl and C₅₋₆cycloalkenyl which group may be optionally substituted by halo, cyano, hydroxy or C₁₋₄alkoxy; R³ and R⁴ are independently hydrogen or a group selected from C₁₋₄alkyl, C₂₋₄alkenyl, C₂₋₄alkynyl, C₃₋₅cycloalkyl, pentenyl, aryl, heteroaryl and heterocyclyl which group is optionally substituted by one or more substituents independently selected from halo, nitro, cyano, trifluoromethyl, trifluoromethyloxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, —OR¹⁸, —SR¹⁹, —SOR¹⁹, —SO₂R¹⁹, —CONR¹⁸R²⁰ and —NR¹⁶COR¹⁸; or R¹ and R³ together with the nitrogen and carbon atoms to which they are respectively attached form a saturated 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl; or R³ and R⁴ together form a carbocyclic or saturated heterocyclic 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl; R⁷ is hydrogen or a group selected from C₁₋₄alkyl, heteroalkyl, C₃₋₅cycloalkyl, aryl, heteroaryl or heterocyclyl where the group is optionally substituted by halo, C₁₋₄alkyl, C₁₋₄alkoxy, C₃₋₅cycloalkyl, heterocyclyl, aryl, heteroaryl or heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₅cycloalkyl, heterocyclyl, C₁₋₄alkoxyC₁₋₄alkyl, haloC₁₋₄alkoxyC₁₋₄alkyl, —COC₁₋₄alkyl, —OR²¹, —CO₂R²¹, —SR²⁵, —SOR²⁵, —SO₂R²⁵, —CONR²¹R²² and —NHCONR²¹R²²; or R³ and R⁷ together with the carbon atoms to which they are each attached and (CR⁵R⁶)ₙ form a saturated carbocyclic or heterocyclic 5- or 6-membered ring; R⁸ is selected from hydrogen, C₁₋₄alkyl and haloC₁₋₄alkyl; R⁹ and R¹⁰ are independently hydrogen, C₁₋₆alkyl or C₃₋₆cycloalkyl; or R⁹ and R¹⁰ together with the nitrogen to which they are attached form a heterocyclic 4 to 6-membered ring. R¹¹ is C₁₋₄alkyl or C₃₋₅cycloalkyl; R¹², R¹³, R¹⁴ and R¹⁵ are independently selected from hydrogen, C₁₋₄alkyl and C₃₋₄cycloalkyl; R¹⁶ is hydrogen or C₁₋₄alkyl; R¹⁷ is selected from halo, C₁₋₄alkyl, C₃₋₅cycloalkyl and C₁₋₄alkoxy; R¹⁸ is hydrogen or a group selected from C₁₋₄alkyl, C₃₋₅cycloalkyl, C₅₋₆cycloalkenyl, saturated heterocyclyl, aryl, heteroaryl, arylC₁₋₄alkyl and heteroarylC₁₋₄alkyl which group is optionally substituted by one or more halo; R¹⁹ and R²⁵ are independently a group selected from C₁₋₄alkyl, C₃₋₅cycloalkyl, C₅₋₆cycloalkenyl, saturated heterocyclyl, aryl, heteroaryl, arylC₁₋₄alkyl and heteroarylC₁₋₄alkyl which group is optionally substituted by one or more halo; R²⁰ is hydrogen, C₁₋₄alkyl or C₃₋₅cycloalkyl; or R¹⁸ and R²⁰ together with the nitrogen to which they are attached form a heterocyclic 4- to 6-membered ring; R²¹ and R²² are independently hydrogen, C₁₋₄alkyl, haloC₁₋₄alkyl, aryl and arylC₁₋₄alkyl; or R²¹ and R²² together with the nitrogen to which they are attached form a heterocyclic 5- to 6-membered ring.

In another aspect of the present invention there is provided a compound of formula (1) or a pharmaceutically acceptable salt thereof.

It is to be understood that, insofar as certain of the compounds of formula (1) defined above may exist in optically active or racemic forms by virtue of one or more asymmetric carbon or sulphur atoms, the invention includes in its definition any such optically active or racemic form which possesses metalloproteinases inhibition activity and in particular TACE inhibition activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Compounds of formula (1) are therefore provided as enantiomers, diastereomers, geometric isomers and atropisomers.

Within the present invention it is to be understood that a compound of formula (1) or a salt thereof may exhibit the phenomenon of tautomerism and that the formulae drawings within this specification can represent only one of the possible tautomeric forms. It is to be understood that the invention encompasses any tautomeric form which has metalloproteinases inhibition activity and in particular TACE inhibition activity and is not to be limited merely to any one tautomeric form utilised within the formulae drawings.

It is also to be understood that certain compounds of formula (1) and salts thereof can exist in solvated as well as unsolvated forms such as, for example, hydrated forms. It is to be understood that the invention encompasses all such solvated forms which have metalloproteinases inhibition activity and in particular TACE inhibition activity.

It is also to be understood that certain compounds of formula (1) may exhibit polymorphism, and that the invention encompasses all such forms which possess metalloproteinases inhibition activity and in particular TACE inhibition activity.

The present invention relates to compounds of formula (1) as defined herein as well as to the salts thereof. Salts for use in pharmaceutical compositions will be pharmaceutically acceptable salts, but other salts may be useful in the production of compounds of formula (1) and their pharmaceutically acceptable salts. Pharmaceutically acceptable salts of the invention may, for example, include acid addition salts of compounds of formula (1) as defined herein which are sufficiently basic to form such salts. Such acid addition salts include but are not limited to hydrochloride, hydrobromide, citrate and maleate salts and salts formed with phosphoric and sulphuric acid. In addition where compounds of formula (1) are sufficiently acidic, salts are base salts and examples include but are not limited to, an alkali metal salt for example sodium or potassium, an alkaline earth metal salt for example calcium or magnesium, or organic amine salts for example triethylamine or tris-(2-hydroxyethyl)amine

The compounds of formula (1) may also be provided as in vivo hydrolysable esters. An in vivo hydrolysable ester of a compound of formula (1) containing a carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

Suitable pharmaceutically-acceptable esters for hydroxy include inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example formyl, acetyl; benzoyl; phenylacetyl; substituted benzoyl and phenylacetyl, C₁₋₁₀alkoxycarbonyl (to give alkyl carbonate esters), for example ethoxycarbonyl; di-(C₁₋₄)alkylcarbamoyl and N-(di-(C₁₋₄)alkylaminoethyl)-N-(C₁₋₄)alkylcarbamoyl (to give carbamates); di-(C₁₋₄)alkylaminoacetyl and carboxyacetyl. Examples of ring substituents on phenylacetyl and benzoyl include aminomethyl, (C₁₋₄)alkylaminomethyl and di-((C₁₋₄)alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)O(C₁₋₆)alkyl-CO—, wherein R^{A} is for example, benzyloxy-(C₁₋₄)alkyl, or phenyl). Suitable substituents on a phenyl group in such esters include, for example, 4-(C₁₋₄)piperazino-(C₁₋₄)alkyl, piperazino-(C₁₋₄)alkyl and morpholino-(C₁₋₄)alkyl.

In this specification the generic term “alkyl” includes both straight-chain and branched-chain alkyl groups. However references to individual alkyl groups such as “propyl” are specific for the straight chain version only and references to individual branched-chain alkyl groups such as tert-butyl are specific for the branched chain version only. For example, “C₁₋₃alkyl” includes methyl, ethyl, propyl and isopropyl, examples of “C₁₋₄alkyl” include the examples of “C₁₋₃alkyl” and butyl and tert-butyl and examples of “C₁₋₆alkyl” include the examples of “C₁₋₄alkyl” and additionally pentyl, 2,3-dimethylpropyl, 3-methylbutyl and hexyl. An analogous convention applies to other generic terms, for example “C₂₋₄alkenyl” includes vinyl, allyl and 1-propenyl and examples of “C₂₋₆alkenyl” include the examples of “C₂₋₄alkenyl” and additionally 1-butenyl, 2-butenyl, 3-butenyl, 2-methylbut-2-enyl, 3-methylbut-1-enyl, 1-pentenyl, 3-pentenyl and 4-hexenyl. Examples of “C₂₋₄alkynyl” includes ethynyl, 1-propynyl, 2-propynyl and 3-butynyl and examples of “C₂₋₆alkynyl” include the examples of “C₂₋₄alkynyl” and additionally 2-pentynyl, hexynyl and 1-methylpent-2-ynyl. Where examples are given of generic terms, these examples are not limiting.

“Cycloalkyl” is a monocyclic, saturated alkyl ring. The term “C₃₋₄cycloalkyl” includes cyclopropyl and cyclobutyl. The term “C₃₋₅cycloalkyl” includes “C₃₋₄cycloalkyl” and cyclopentyl. The term “C₃₋₆cycloalkyl” includes “C₃₋₅cycloalkyl” and cyclohexyl. The term “C₃₋₇cycloalkyl” includes “C₃₋₆cycloalkyl” and additionally cycloheptyl. The term “C₃₋₁₀cycloalkyl” includes “C₃₋₇cycloalkyl” and additionally cyclooctyl, cyclononyl and cyclodecyl.

“Cycloalkenyl” is a monocyclic ring containing 1, 2, 3 or 4 double bonds. Examples of “C₃₋₇cycloalkenyl”, “C₅₋₇cycloalkenyl” and “C₅₋₆cycloalkenyl” are cyclopentenyl, cyclohexenyl and cyclohexadiene and examples of “C₅₋₁₀cycloalkenyl” include these examples and cyclooctatriene.

Unless otherwise specified “aryl” is monocyclic or bicyclic. Examples of “aryl” therefore include phenyl (an example of monocyclic aryl) and naphthyl (an example of bicyclic aryl).

Examples of “arylC₁₋₄alkyl” are benzyl, phenethyl, naphthylmethyl and naphthylethyl.

Unless otherwise specified “heteroaryl” is a monocyclic or bicyclic aryl ring containing 5 to 10 ring atoms of which 1, 2, 3 or 4 ring atoms are chosen from nitrogen, sulphur or oxygen where a ring nitrogen or sulphur may be oxidised. Examples of heteroaryl are pyridyl, imidazolyl, quinolinyl, cinnolyl, pyrimidinyl, thienyl, pyrrolyl, pyrazolyl, thiazolyl, oxazolyl, isoxazolyl, pyrazinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl and pyrazolopyridinyl. Preferably heteroaryl is pyridyl, imidazolyl, quinolinyl, pyrimidinyl, thienyl, pyrazolyl, thiazolyl, oxazolyl and isoxazolyl. More preferably heteroaryl is pyridyl, imidazolyl and pyrimidinyl. Examples of “monocyclic heteroaryl” are pyridyl, imidazolyl, pyrimidinyl, thienyl, pyrrolyl, pyrazolyl, thiazolyl, oxazolyl, isoxazolyl and pyrazinyl. Examples of “bicyclic heteroaryl” are quinolinyl, quinazolinyl, cinnolinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl and pyrazolopyridinyl. Preferred examples B when B is heteroaryl are those examples of bicyclic heteroaryl.

Examples of “heteroarylC₁₋₄alkyl” are pyridylmethyl, pyridylethyl, pyrimidinylethyl, pyrimidinylpropyl, pyrimidinylbutyl, imidazolylpropyl, imidazolylbutyl, quinolinylpropyl, 1,3,4-triazolylpropyl and oxazolylmethyl.

“Heterocyclyl” is a saturated, partially saturated or unsaturated, monocyclic or bicyclic ring (unless otherwise stated) containing 4 to 12 atoms of which 1, 2, 3 or 4 ring atoms are chosen from nitrogen, sulphur or oxygen, which may, unless otherwise specified, be carbon or nitrogen linked, wherein a —CH₂— group can optionally be replaced by a —C(O)—; and where unless stated to the contrary a ring nitrogen or sulphur atom is optionally oxidised to form the N-oxide or S-oxide(s); a ring —NH is optionally substituted by acetyl, formyl, methyl or mesyl; and a ring is optionally substituted by one or more halo. Examples and suitable values of the term “heterocyclyl” are piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, pyranyl, dihydro-2H-pyranyl, tetrahydrofuranyl, 2,5-dioximidazolidinyl, 2,2-dimethyl-1,3-dioxolanyl and 3,4-methylenedioxyphenyl. Preferred values are 3,4-dihydro-2H-pyran-5-yl, tetrahydrofuran-2-yl, 2,5-dioximidazolidinyl, 2,2-dimethyl-1,3-dioxolan-2-yl, 2,3-methylenedioxyphenyl and 3,4-methylenedioxyphenyl. Other values are pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinoline, tetrahydroisoquinoline and isoindolinyl. Examples of monocyclic heterocyclyl are piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, pyranyl, tetrahydrofuranyl, 2,5-dioximidazolidinyl and 2,2-dimethyl-1,3-dioxolanyl. Examples of bicyclic heterocyclyl are pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl, isoindolinyl, 2,3-methylenedioxyphenyl, and 3,4-dimethylenedioxyphenyl. Examples of saturated heterocyclyl are piperidinyl, pyrrolidinyl and morpholinyl.

The term “halo” refers to fluoro, chloro, bromo and iodo.

Examples of “C₁₋₃alkoxy” and “C₁₋₄alkoxy” include methoxy, ethoxy, propoxy and isopropoxy. Examples of “C₁₋₆alkoxy” include the examples of “C₁₋₄alkoxy” and additionally pentyloxy, 1-ethylpropoxy and hexyloxy.

“Heteroalkyl” is alkyl containing at least one carbon atom and having at least one carbon atom replaced by a hetero group independently selected from N, O, S, SO, SO₂, (a hetero group being a hetero atom or group of atoms). Examples include —CH₂O—, OCH₂—, —CH₂CH₂O—, —CH₂SCH₂CH₂ and —OCH(CH₃)₂—.

“HaloC₁₋₄alkyl” is a C₁₋₄alkyl group substituted by one or more halo. Examples of “haloC₁₋₄alkyl” include fluoromethyl, trifluoromethyl, 1-chloroethyl, 2-chloroethyl, 2-bromopropyl, 1-fluoroisopropyl and 4-chlorobutyl. Examples of “haloC₁₋₆alkyl” include the examples of “haloC₁₋₄alkyl” and 1-chloropentyl, 3-chloropentyl and 2-fluorohexyl.

Examples of “hydroxyC₁₋₄alkyl” include hydroxymethyl, 1-hydroxyethyl, 2-hydroxyethyl, 2-hydroxypropyl, 1-hydroxyisopropyl and 4-hydroxybutyl.

Example of “C₁₋₄alkoxyC₁₋₄alkyl” include methoxymethyl, ethoxymethyl, methoxyethyl, methoxypropyl and propoxybutyl.

“HaloC₁₋₄alkoxyC₁₋₄alkyl” is a C₁₋₄alkoxyC₁₋₄alkyl group substituted by one or more halo. Examples of “haloC₁₋₄alkoxyC₁₋₄alkyl” include trifluoromethoxymethyl, 1-(chloromethoxy)ethyl, 2-fluoroethoxymethyl, 2-(4-bromobutoxy)ethyl and 2-(2-iodoethoxy)ethyl.

Examples of “carboxyC₁₋₄alkyl” include carboxymethyl, 2-carboxyethyl and 2-carboxypropyl.

A “carbocyclic 5 to 6-membered” ring is (unless specifically stated) a saturated, partially saturated or unsaturated ring containing 5 to 6 ring carbon atoms. Examples include cyclopentyl, cyclopent-3-enyl, cyclohexyl and cyclopent-2-enyl. An analogous convention applies for a “carbocyclic 3 to 7-membered” ring which includes the examples a “carbocyclic 5 to 6-membered” ring and additionally cylopropyl and cyclobutyl.

Heterocyclic rings are rings containing 1, 2 or 3 ring atoms selected from nitrogen, oxygen and sulphur. “Heterocyclic 4 to 6-membered”, “heterocyclic 5 to 6-membered” and “heterocyclic 5 to 7-membered” rings are pyrrolidinyl, piperidinyl, piperazinyl, homopiperidinyl, homopiperazinyl, thiomorpholinyl, thiopyranyl and morpholinyl. “Heterocyclic 4 to 7-membered” rings include the examples of “heterocyclic 5 to 7-membered” and additionally azetidinyl. Saturated heterocyclic 3- to 7-membered, 4- to 7-membered and 5- to 6-membered rings include piperidinyl, pyrrolidinyl and morpholinyl.

Where optional substituents are chosen from “one of more” groups or substituents it is to be understood that this definition includes all substituents being chosen from one of the specified groups or the substituents being chosen from two or more of the specified groups. Preferably “one or more” means “1, 2 or 3” and this is particularly the case when the group or substituent is halo. “One or more” may also means “1 or 2”.

Compounds of the present invention have been named with the aid of computer software (ACD/Name version 5.09).

Preferred values of Y¹, Y², z, n, W, m, D, X, B, R³, R⁴, R⁵, R⁶ and R⁷ are as follows. Such values may be used where appropriate with any of the definitions, claims or embodiments defined herein.

In one aspect of the invention Y¹ and Y² are both O.

In one aspect of the invention z is NR⁸.

In one aspect of the invention n is 1. In another aspect n is 0.

In one aspect of the invention W is NR¹. In another aspect W is CR¹R². In a further aspect W is a bond.

In one aspect of the invention m is 0. In another aspect m is 1.

In one aspect of the invention D is hydrogen, methyl or fluoro. In another aspect D is hydrogen.

In one aspect of the invention X is —CR¹²R¹³-Q- or —CR¹²R¹³-Q-CR¹⁴R¹⁵—. In another aspect of the invention X is —CR¹²R¹³-Q-, -Q-CR¹⁴R¹⁵— or —CR¹²R¹³-Q-CR¹⁴R¹⁵—. In another aspect X is Q. In a further aspect X is —(CH₂)—O—, —O—(CH₂)—, —(CH₂)—O—(CH₂)— or —(CHMe)—O— or O. In yet another aspect X is —(CH₂)—O— or —O—(CH₂)—

In one aspect of the invention Q is O.

In one aspect, when n is 1 and W is NR¹, CR¹R² or a bond; or when n is 0 and W is CR¹R²; B is a group selected from aryl, heteroaryl and heterocyclyl, where each group is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloalkyl or heterocyclyl. In another aspect, when n is 1 and W is NR¹, CR¹R² or a bond; or when n is 0 and W is CR¹R²; B is phenyl, naphthyl, pyridyl, quinolinyl, isoquinolinyl, thienopyridyl, naphthyridinyl, 2,3-methylenedioxyphenyl, 3,4-methylenedioxyphenyl, thienopyrimidinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl or isoindolinyl, where each is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl. In another aspect when n is 1 and W is NR¹, CR¹R² or a bond; or when n is 0 and W is CR¹R²; B is phenyl, naphthyl, pyridyl, quinolinyl, isoquinolinyl, thienopyridyl, naphthyridinyl, 2,3-methylenedioxyphenyl, 3,4-methylenedioxyphenyl, thienopyrimidinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl or isoindolinyl,where each is optionally substituted by one or more groups independently selected from trifluoromethyl, trifluoromethoxy, fluoro, chloro, bromo, methyl, isopropyl, ethynyl, cyano, acetamido, propyloxy, isopropyloxymethoxy, nitro, pyrrolidinylcarbonyl, N-propylcarbamoyl, pyrrolidinyl, piperidinyl, isoxazolyl, pyrazolyl, imidazolyl, oxazolyl, thiazolyl, pyrimidinyl and pyridyl; or B is vinyl or ethynyl optionally substituted by methyl or ethyl. In a further aspect when n is 1 and W is NR¹, CR¹R² or a bond; or when n is 0 and W is CR¹R²; B is quinolin-4-yl, naphthyl, 2-methylquinolin-4-yl, 3-methylnaphthyl, 7-methylquinolin-5-yl, 6-methylquinolin-8-yl, 7-methylisoquinolin-5-yl, 6-methylthieno[2,3-b]pyridyl, 5-methylthieno[3,2-b]pyridyl, 2-methyl-1,8-naphthyridinyl, 2-trifluoromethylquinolin-4-yl, 2-ethynylquinolin-4-yl, 7-chloroquinolin-5-yl, 7-fluoro-2-methylquinolin-4-yl, 2-methyl-N-oxoquinolin-4-yl, 3-methylisoquinolin-1-yl, 5-fluoro-2-methylquinolin-4-yl, 2,6-dimethylpyrid-4-yl, 2,5-dimethylpyridin-4-yl, 2,5-dimethylphenyl, 2,5-difluorophenyl, 3,5-difluorophenyl, 2,6-difluoro-3-methylphenyl, 2-chloro-6-fluorophenyl, 3-fluoro-6-methylphenyl, 2,6-difluorophenyl, 3,4-dichlorophenyl, 2-fluoro-3-methylphenyl, 2,4-dichlorophenyl, 2,6-dichlorophenyl, 2,4,6-trimethylphenyl, 3,4-dimethylphenyl, 3,5-dimethylphenyl, 3-chloro-4-methylphenyl, 2,3-methylenedioxyphenyl, 3,4-methylenedioxyphenyl, 5-fluoro-2-methylpyridinyl, 2,4-dimethylphenyl, 1-methylquinolinyl, 2-chloro-4-fluorophenyl, 2-chloro-4-trifluoromethylphenyl, 2-bromo-4,6-difluorophenyl, 2-bromo-4-fluorophenyl, 2,4-dichlorophenyl, 2-bromo-4-chlorophenyl, 2-methoxy-4-methylphenyl, 4-chloro-2-nitrophenyl, 4-methyl-2-nitrophenyl, 2,4-difluorophenyl, 4-bromo-2-fluorophenyl, 2-methoxy-4-nitrophenyl, 2-chloro-4-nitrophenyl, 4-bromo-2-methoxyphenyl, 2-fluoro-4-nitrophenyl, 2-chloro-4-bromophenyl, 2-chloro-4-methylphenyl, 2-chloro-4-methoxyphenyl, 4-fluoro-2-methoxyphenyl, 2-fluoro-4-chlorophenyl, 4-fluoro-2-methylphenyl, 7-chloroquinolin-4-yl, 8-chloroquinolin-4-yl, 3-chloro-5-trifluoromethylpyrid-2-yl, 3,5-dichloropyrid-2-yl, 6-chloroquinolin-4-yl, 5-methylthieno[2,3-d]pyrimidin-4-yl, 7-methylthieno[3,2-d]pyrimidin-4-yl, 8-fluoroquinolin-4-yl, 4-chloro-2-(isoxazol-5-yl)phenyl, 2-(isoxazol-5-yl)-4-trifluoromethylphenyl, 6-fluoroquinolin-4-yl, 2-methylquinolin-4-yl, 6-chloro-2-methylquinolin-4-yl, 1,6-naphthyridin-4-yl, thieno[3,2-b]pyrid-7-yl, 5-fluoro-2-(isoxazol-5-yl)phenyl, 4-fluoro-2-(isoxazol-5-yl)phenyl, 4-chloro-2-trifluoromethylphenyl, 2-chloro-5-fluorophenyl, vinyl, ethynyl, prop-1-enyl, prop-1-ynyl or but-1-ynyl. In one aspect, B is a group selected from bicyclic aryl, bicyclic heteroaryl and bicyclic heterocyclyl, where each group is optionally substituted by one or more groups independently selected from nitro, trifluoromethyl, trifluoromethoxy, halo, C₁₋₄alkyl (optionally substituted by one or more halo), C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloalkyl or heterocyclyl. In another aspect B is naphthyl, quinolinyl, isoquinolinyl, thienopyridyl, 2,3-methylenedioxyphenyl, 3,4-methylenedioxyphenyl, naphthyridinyl, thienopyrimidinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl or isoindolinyl, where each is optionally substituted by one or more groups independently selected from nitro, trifluoromethyl, trifluoromethoxy, halo, C₁₋₄alkyl(optionally substituted by one or more halo), C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl. In another aspect B is naphthyl, quinolinyl, isoquinolinyl, thienopyridyl, 2,3-methylenedioxyphenyl, 3,4-methylenedioxyphenyl, naphthyridinyl, thienopyrimidinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl or isoindolinyl,where each is optionally substituted by one or more groups independently selected from trifluoromethyl, trifluoromethoxy, fluoro, chloro, bromo, methyl, isopropyl, ethynyl, cyano, acetamido, propyloxy, isopropyloxy, methoxy, nitro, pyrrolidinylcarbonyl, N-propylcarbamoyl; or B is vinyl or ethynyl optionally substituted by methyl or ethyl. In another aspect B is quinolin-4-yl, naphthyl, 2-methylquinolin-4-yl, 3-methylnaphthyl, 7-methylquinolin-5-yl, 6-methylquinolin-8-yl, 7-methylisoquinolin-5-yl, 6-methylthieno[2,3-b]pyridyl, 5-methylthieno[3,2-b]pyridyl, 2-methyl-1,8-naphthyridinyl, 2-trifluoromethylquinolin-4-yl, 2-ethynylquinolin-4-yl, 7-chloroquinolin-5-yl, 7-fluoro-2-methylquinolin-4-yl, 2-methyl-N-oxoquinolin-4-yl, 3-methylisoquinolin-1-yl, 5-fluoro-2-methylquinolin-4-yl, 3,4-methylenedioxyphenyl, 1-methylquinolinyl, 7-chloroquinolin-4-yl, 8-chloroquinolin-4-yl, 6-chloroquinolin-4-yl, 5-methylthieno[2,3-d]pyrimidin-4-yl, 7-methylthieno[3,2-d]pyrimidin-4-yl, 8-fluoroquinolin-4-yl, 6-fluoroquinolin-4-yl, 2-methylquinolin-4-yl, 6-chloro-2-methylquinolin-4-yl, 1,6-naphthyridin-4-yl, thieno[3,2-b]pyrid-7-yl, vinyl, ethynyl, prop-1-enyl, prop-1-ynyl or but-1-ynyl. In another aspect B is a group selected from aryl and heteroaryl where each group is optionally substituted by one or more groups independently selected from halo, C₁₋₄alkyl (optionally substituted by one or more halo), heteroaryl and C₂₋₄alkynyl. In another aspect B is a group selected from quinolinyl, pyridyl and phenyl where each group is optionally substituted by one or more methyl, trifluoromethyl, trifluoromethoxy, halo or isoxazolyl. In a further aspect B is 2-methylquinolin-4-yl, 2,5-dimethylphenyl or 2,5-dimethylpyrid-4-yl. In yet another aspect B is 2-methylquinolin-4-yl.

In one aspect of the invention R¹ is hydrogen or methyl. In another aspect R¹ is hydrogen.

In one aspect of the invention R2 is hydrogen or methyl. In another aspect R² is hydrogen.

In one aspect of the invention R³ is hydrogen, methyl, ethyl, propyl or phenyl. In another aspect R³ is hydrogen.

In one aspect of the invention R⁴ is hydrogen or methyl. In another aspect R⁴ is hydrogen.

In one aspect of the invention R⁵ is hydrogen or methyl. In another aspect R⁵ is hydrogen.

In one aspect of the invention R⁶is hydrogen or methyl. In another aspect R⁶ is hydrogen.

In one aspect of the invention R¹ and R³ together with the nitrogen or carbon and carbon to which they are respectively attached form a saturated 3- to 7-membered ring optionally containing 1 or 2 heteroatom groups selected from NH, O, S, SO and SO₂ where the ring is optionally substituted by one or more C₁₋₄alkyl. In another aspect R¹ and R³ together with the nitrogen or carbon and carbon to which they are respectively attached form a piperidine, pyrrolidine, piperazine, morpholine, cyclohexane or cyclopentane ring.

In one aspect of the invention R³ and R⁴ together form a saturated 3- to 7-membered ring optionally containing 1 or 2 heteroatom groups selected from NH, O, S, SO and SO₂ where the ring is optionally substituted by one or more C₁₋₄alkyl.

In one aspect of the invention R⁵ and R⁶ together form a saturated 3- to 7-membered ring optionally containing 1 or 2 heteroatom groups selected from NH, O, S, SO and SO₂ where the ring is optionally substituted by one or more C₁₋₄alkyl.

In one aspect of the invention R⁷ is hydrogen or a group selected from C₁₋₄alkyl, C₃₋₅cycloalkyl, aryl, heteroaryl or heterocyclyl where the group is optionally substituted by heterocyclyl, aryl and heteroaryl; and wherein the group from which R⁷ may be selected is optionally substituted on the group and/or on its optional substituent by one or more substituents independently selected from halo, cyano, C₁₋₄alkyl, —OR²¹, —CO₂R²¹, —NR²¹COR²², —NR²¹CO₂R²² and —CONR²¹R²². In one aspect R⁷ is hydrogen or a group selected from C₁₋₄alkyl, C₃₋₅cycloalkyl, aryl, heteroaryl or heterocyclyl where the group is optionally substituted by heterocyclyl, aryl and heteroaryl; and wherein the group from which R⁷ may be selected is optionally substituted on the group and/or on its optional substituent by one or more substituents independently selected from halo, cyano, C₁₋₄alkyl, —OR²¹, —CO₂R²¹, and NR²¹CO₂R²². In another aspect R⁷ is hydrogen or a group selected from C₁₋₄alkyl, arylC₁₋₄alkyl, heteroarylC₁₋₄alkyl, heterocyclylC₁₋₄alkyl, aryl, heteroaryl, heterocyclyl and C₃₋₅cycloalkyl where the group is optionally substituted by cyano, C₁₋₄alkyl, halo, —OR²¹, —NR²¹R²², —CO₂R²¹ and —NR²¹CO₂R²². In a further aspect R⁷ is selected from hydrogen, methyl, ethyl, propyl, isopropyl, cyclopropyl, butyl, tert-butyl, isobutyl, 1-hydroxyethyl, 2-hydroxyethyl, 3-hydroxypropyl, methoxymethyl, 2-methoxyethyl, 2-cyanoethyl, 2-aminoethyl, phenyl, pyridyl, benzyl, 3-methylbenzyl, phenylethyl, 4-chlorophenylethyl, 4-fluorophenylethyl, phenylpropyl, 4-chlorophenylpropyl, 4-fluorophenylpropyl, 4-methylpiperazin-1-ylethyl, morpholin-4-ylpropyl, pyrimidin-2-ylethyl, pyrimidin-2-ylpropyl, pyrimidin-2-ylbutyl, 5-fluoropyrimidin-2-ylpropyl, imidazol-1-ylpropyl, imidazol-1-ylbutyl, 1,3,4-triazolylpropyl, piperidinyl, tetrahydro-2H-pyranyl, tetrahydro-2H-pyranylmethyl, pyrid-2-ylmethyl, pyrid-4-ylmethyl, pyrid-3-ylmethyl, piperidin-4-ylmethyl, N-(tert-butoxycarbonyl)piperidin-4-yl, tert-butoxycarbonylaminomethyl, N-(methylcarbonyl)piperidin-4-yl), benzyloxyethyl, N-(tert-butoxycarbonyl)piperidin-4-ylmethyl, (3,4,4-trimethyl-2,5-dioximidazolidin-1-yl)methyl and N-benzoyl-N-phenylaminomethyl. In a further aspect R⁷ is hydrogen or C₁₋₄alkyl optionally substituted with halo, hydroxy or C₁₋₃alkoxy. In yet another aspect R⁷ is hydrogen, methyl or ethyl.

In one aspect of the invention R³ and R⁷ together with the carbon atoms to which they are each attached and (CR⁵R⁶)ₙ form a saturated 5- to 7-membered ring optionally containing a heteroatom group selected from NH, O, S, SO and SO₂ where the ring is optionally substituted on carbon or nitrogen by one or more C₁₋₄alkyl. In another aspect R³ and R⁷ together with the carbon atoms to which they are each attached and (CR⁵R⁶)ₙ form a piperidinyl, pyrrolidinyl, piperazine, morpholine, cyclohexane or cyclopentane ring.

In one aspect of the invention R⁸ is hydrogen or methyl. In another aspect R⁸ is hydrogen.

In one aspect R⁹ is hydrogen or methyl.

In one aspect R¹⁰ is hydrogen or methyl.

In one aspect R¹¹ is methyl.

In one aspect R¹² is hydrogen or methyl.

In one aspect R¹³ is hydrogen or methyl.

In one aspect R¹⁴ is hydrogen or methyl.

In one aspect R¹⁵ is hydrogen or methyl.

In one aspect R¹⁶ is hydrogen or methyl.

In one aspect R¹⁷ is selected from fluoro, chloro, methyl or methoxy.

In one aspect of the invention R¹⁹ is a group selected from C₁₋₆alkyl, aryl and arylC₁₋₄alkyl which group is optionally substituted by halo. In another aspect R¹⁹ is a group selected from methyl, phenyl and benzyl which group is optionally substituted by chloro. In one aspect of the invention R¹⁹ is methyl.

In one aspect of the invention R¹⁸ is hydrogen or a group selected from C₁₋₆alkyl, aryl and arylC₁₋₄alkyl which group is optionally substituted by halo. In another aspect R¹⁸ is hydrogen or a group selected from methyl, phenyl and benzyl which group is optionally substituted by chloro.

In one aspect R²⁰ is hydrogen or methyl.

In one aspect R²¹ is hydrogen, methyl, ethyl, phenyl and benzyl.

In one aspect R²² is hydrogen, methyl, ethyl, tert-butyl, phenyl and benzyl. In another aspect R²² is hydrogen or methyl.

In one aspect of the invention R²¹ and R²² are independently hydrogen, C₁₋₄alkyl, haloC₁₋₄alkyl, aryl, arylC₁₋₄alkyl or benzoyl.

In one aspect of the invention R²⁵ is a group selected from C₁₋₆alkyl, aryl and arylC₁₋₄alkyl which group is optionally substituted by halo. In another aspect R²⁵ is a group selected from methyl, phenyl and benzyl which group is optionally substituted by chloro. In one aspect of the invention R²⁵ is methyl.

A preferred class of compound is of formula (1) wherein: Y¹ and Y² are both O.
z is NR⁸;
n is 1 and W is NR¹, CR¹R²or a bond; or n is 0 and W is CR¹R²;
m is 1;
D is hydrogen, methyl or fluoro;
X is —CR¹²R¹³-Q-, -Q-CR¹⁴R¹⁵—, —CR¹²R¹³-Q-CR¹⁴R¹⁵— or Q; Q is O;
B is a group selected from aryl, heteroaryl and heterocyclyl, where each group is optionally substituted by one or more groups independently selected from nitro, trifluoromethyl, trifluoromethoxy, halo, cyano, C₁₋₄alkyl (optionally substituted one or more halo), C₂₋₄alkenyl, heteroaryl, —OR⁹, —NR⁹R¹⁰, —CONR⁹R¹⁰ and —NR⁹COR¹⁰; or B is C₂₋₄alkenyl or C₂₋₄alkynyl, optionally substituted by C₁₋₄alkyl, C₃₋₆cycloalkyl or heterocyclyl; R¹ and R² are independently hydrogen or methyl; R³ is hydrogen, methyl, ethyl, propyl or phenyl; R⁴, R⁵, R⁶, R⁸, R⁹, R¹⁰, R¹², R¹³, R¹⁴and R¹⁵ are independently hydrogen or methyl; R⁷ is hydrogen or a group selected from C₁₋₄alkyl, C₃₋₅cycloalkyl, aryl, heteroaryl or heterocyclyl where the group is optionally substituted by heterocyclyl, aryl and heteroaryl; and wherein the group from which R⁷ may be selected is optionally substituted on the group and/or on its optional substituent by one or more substitutents independently selected from halo, cyano, C₁₋₄alkyl, —OR²¹, —CO₂R²¹, —NR²¹COR²², —NR²¹CO₂R²² and —CONR²¹R²²; R²¹ is hydrogen, methyl, ethyl, phenyl or benzyl; R²² is hydrogen, methyl, ethyl, tert-butyl, phenyl or benzyl.

Another preferred class of compound is of formula (1) wherein:
Y¹ and Y² are both O;
z is NR⁸;
n is 1 and W is NR¹, CR¹R²or a bond; or n is 0 and W is CR¹R²;
m is 1;
D is hydrogen, methyl or fluoro;
X is —CR¹²R¹³-Q-, -Q-CR¹⁴R¹⁵—, —CR¹²R¹³-Q-CR¹⁴R¹⁵— or Q; Q is O;
B is phenyl, naphthyl, pyridyl, quinolinyl, isoquinolinyl, thienopyridyl, naphthyridinyl, 2,3-methylenedioxyphenyl, 3,4-methylenedioxyphenyl, thienopyrimidinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl or isoindolinyl,where each is optionally substituted by one or more groups independently selected from trifluoromethyl, trifluoromethoxy, fluoro, chloro, bromo, methyl, isopropyl, ethynyl, cyano, acetamido, propyloxy, isopropyloxymethoxy, nitro, pyrrolidinylcarbonyl, N-propylcarbamoyl, pyrrolidinyl, piperidinyl, isoxazolyl, pyrazolyl, imidazolyl, oxazolyl, thiazolyl, pyrimidinyl and pyridyl; or B is vinyl or ethynyl optionally substituted by methyl or ethyl. R¹ and R² are independently hydrogen or methyl; R³ is hydrogen, methyl, ethyl, propyl or phenyl; R⁴, R⁵, R⁶, R⁸, R¹², R¹³, R¹⁴ and R¹⁵ are independently hydrogen or methyl; R⁷ is hydrogen or a group selected from C₁₋₄alkyl, arylC₁₋₄alkyl, heteroarylC₁₋₄alkyl, heterocyclylC₁₋₄alkyl, aryl, heteroaryl, heterocyclyl and C₃₋₅cycloalkyl where the group is optionally substituted by cyano, C₁₋₄alkyl, halo, —OR²¹, —NR²¹R²², —CO₂R²¹ and —NR²¹CO₂R²²; R²¹ is hydrogen, methyl, ethyl, phenyl and benzyl; R²² is hydrogen, methyl, ethyl, phenyl, tert-butyl and benzyl.

Another preferred class of compound is of formula (1) wherein:
Y¹ and Y² are both O;
z is NR⁸;
n is 0;
W is NR¹;
m is 1;
D is hydrogen, methyl or fluoro;
X is —CR¹²R¹³-Q-, -Q-CR¹⁴R¹⁵—, —CR¹²R¹³-Q-CR¹⁴R¹⁵— or Q; Q is O;
B is a group selected from bicyclic aryl, bicyclic heteroaryl and bicyclic heterocyclyl, where each group is optionally substituted by one or more groups independently selected from nitro, trifluoromethyl, trifluoromethoxy, halo, cyano, C₁₋₄alkyl (optionally substituted by one or more halo), C₂₋₄alkenyl, heteroaryl, —OR⁹, —NR⁹R¹⁰, —CONR⁹R¹⁰ and —NR⁹COR¹⁰; or B is C₂₋₄alkenyl or C₂₋₄alkynyl, optionally substituted by C₁₋₄alkyl, C₃₋₆cycloalkyl or heterocyclyl; R¹ is hydrogen; R³ is hydrogen, methyl, ethyl, propyl or phenyl; R⁴, R⁸, R⁹, R¹⁰, R¹², R¹³, R¹⁴ and R¹⁵ are independently hydrogen or methyl; R⁷ is hydrogen or a group selected from C₁₋₄alkyl, arylC₁₋₄alkyl, heteroarylC₁₋₄alkyl, heterocyclylC₁₋₄alkyl, aryl, heteroaryl, heterocyclyl and C₃₋₅cycloalkyl where the group is optionally substituted by cyano, C₁₋₄alkyl, halo, —OR²¹, —NR²¹R²², —CO₂R²¹ and —NR²¹CO₂R²²; R²¹ is hydrogen, methyl, ethyl, phenyl and benzyl; R²² is hydrogen, methyl, ethyl, phenyl, tert-butyl and benzyl.

Another preferred class of compound is of formula (1) wherein:
Y¹ and Y² are both O;
z is NR⁸;
n is 0 or 1;
W is NR¹, CR¹R² or a bond;
m is 1;
D is hydrogen, methyl or fluoro;
X is —CR¹²R¹³-Q-, -Q-CR¹⁴R¹⁵— or —CR¹²^{R}¹³-Q-CR¹⁴R¹⁵—; Q is O;
B is 2-methylquinolin-4-yl;
R¹ and R² are independently hydrogen or methyl;
R³ is hydrogen, methyl, ethyl or phenyl;
R⁴, R⁵, R⁶, R⁸, R¹², R¹³, R¹⁴ and R¹⁵ are independently hydrogen or methyl;
R⁷ is hydrogen or C₁₋₄alkyl optionally substituted with halo, hydroxy or C₁₋₃alkoxy.

In another aspect of the invention, preferred compounds of the invention are any one of: 5-[({4-[(2,5-dimethylbenzyl)oxy]piperidin-1-yl}sulphonyl)methyl]-5-methylimidazolidine-2,4-dione; and 5-[({4-(2-methylquinolin-4-ylmethoxy)piperidin-1-yl}sulphonyl)methyl]-5-methylimidazolidine-2,4-dione.

In another aspect of the invention, preferred compounds are any one of: R/S-5-[({4-(2-methylquinolin-4-ylmethoxy)piperidin-1-yl}sulphonyl)methyl]-5-methylimidazolidine-2,4-dione; 5-[2-({4-[(2-methylquinolin-4-yl)methoxy]piperidin-1-yl}sulphonyl)ethyl]imidazolidine-2,4-dione; 5-{2-[(4-{[(2-methylquinolin-4-yl)oxy]methyl}piperidin-1-yl)sulphonyl]ethyl}imidazolidine-2,4-dione; 5-methyl-5-{[(4-{[(2-methylquinolin-4-yl)oxy]methyl}piperidin-1-yl)sulphonyl]methyl}imidazolidine-2,4-dione; 5-ethyl-5-[({4-[(2-methylquinolin-4-yl)methoxy]piperidin-1-yl}sulphonyl)methyl]imidazolidine-2,4-dione; 5-methyl-5-[2-({4-[(2-methylquinolin-4-yl)methoxy]piperidin-1-yl}sulphonyl)ethyl]imidazolidine-2,4-dione; 5-ethyl-5-[2-({4-[(2-methylquinolin-4-yl)methoxy]piperidin-1-yl}sulphonyl)ethyl]imidazolidine-2,4-dione; (5S)-5-methyl-5-{4-[(2-methylquinolin-4-yl)methoxymethyl]piperidylsulphonylmethyl}-2,4-dioxoimidazolidine; and (5S)-5-ethyl-5-{4-[(2-methylquinolin-4-yl)methoxymethyl]piperidylsulphonylmethyl}-2,4-dioxoimidazolidine.

In another aspect the present invention provides a process for the preparation of a compound of formula (1) or a pharmaceutically acceptable salt or in vivo hydrolysable ester thereof which comprises: a) converting a ketone or aldehyde of formula (2) into a compound of formula (1); 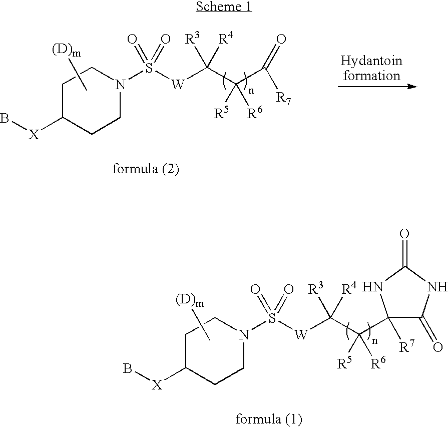
and thereafter if necessary: i) converting a compound of formula (1) into another compound of formula (1); ii) removing any protecting groups; iii) forming a pharmaceutically acceptable salt or in vivo hydrolysable ester.

The hydantoin can be prepared by a number of methods for example; a) The aldehyde or ketone may be reacted with ammonium carbonate and potassium cyanide in aqueous alcohols using the method of Bucherer and Bergs (Adv. Het. Chem., 1985, 38, 177). b) The aldehyde or ketone can be first converted to the cyanohydrin and then further reacted with ammonium carbonate (Chem. Rev, 1950, 56, 403). c) The aldehyde or ketone can be converted to the alpha-amino nitrile and then either reacted with ammonium carbonate or aqueous carbon dioxide or potassium cyanate followed by mineral acid (Chem. Rev, 1950, 56, 403).

The process may further comprise a process for the preparation of a ketone or aldehyde of formula (2) where W is a bond and n is 0 (indicated as a compound of formula (2′)) which process comprises reacting a sulphonamide of formula (3) with a compound of formula (4) where LG represents a leaving group such as halo, alkoxy or aryloxy.

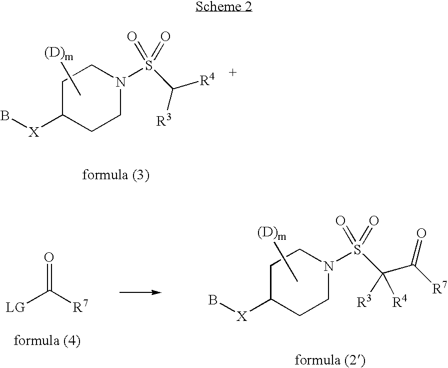

This process comprises the reaction of the sulphonamide of formula (3) with a base such as lithium bis(trimethylsilyl)amide or lithium diisopropylamide in an inert solvent such as tetrahydrofuran at temperatures from −78° C. to 0° C. for 1 to 2 hours followed by addition of a compound of formula (4) at a temperature of −78° C. to room temperature for 1 to 24 hours. A compound of formula (4) is commercially available or can be easily prepared by the skilled man.

A ketone of formula (2′) may additionally be prepared by the process illustrated in Scheme 3:

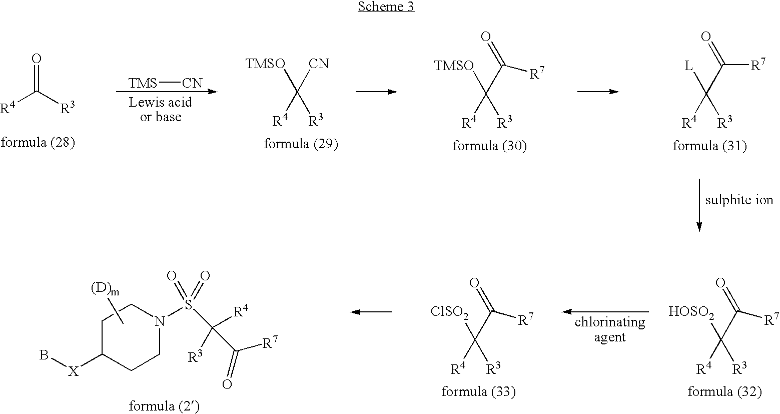

The silyl group present in the compound of formula (30) can be removed by tetrabutylammonium fluoride. Suitable leaving groups (L) are halo, mesyl and tosyl. A suitable chlorinating agent is POCl₃. A compound of formula (2′) is prepared in the last stage by reacting the compound of formula (33) with the appropriate piperidine reagent. A compound of formula (28) is commercially available or can be easily prepared by the skilled person.

Alternatively a process for the preparation of a ketone or aldehyde of formula (2) where W is a bond and n is 1 (indicated as a compound of formula (2″)) comprises reacting a sulphonamide of formula (3) with a compound of formula (5) (an epoxide or equivalent) to give an alcohol of formula (6) and oxidising the alcohol to give a ketone or aldehyde of formula (2″):

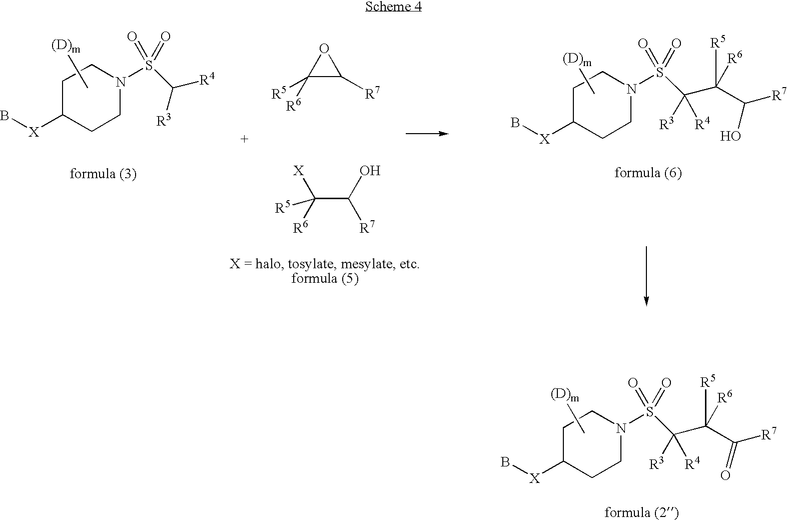

More specifically the process of Scheme 4 comprises the steps of: a) reacting the sulphonamide of formula (3) with a base such as lithium diisopropylamide or lithium bis(trimethylsilyl)amide in tetrahydrofuran at a temperature of −78° C. to 0° C. for 1 to 2 hours followed by addition of an epoxide or equivalent of formula (5) and reaction for 1 to 24 hours at a temperature of −78° C. to room temperature to give an alcohol of formula (6); and b) oxidation of an alcohol of formula (6) to a ketone or aldehyde of formula (2″), suitable reagents are manganese dioxide, pyridinium chlorochromate, pyridinium dichromate or dimethyl sulphoxide/oxalyl chloride/triethylamine. The epoxide or equivalent of formula (5) is commercially available or can be easily prepared by the skilled person.

In another aspect of the invention there is provided a process for the preparation of a compound of formula (1) where W is NR¹, R¹ is hydrogen and n is 0 (indicated as a compound of formula (1′)) which process comprises reaction of a sulphamoyl chloride derivative of formula (7) with an amino-hydantoin derivative of formula (8).

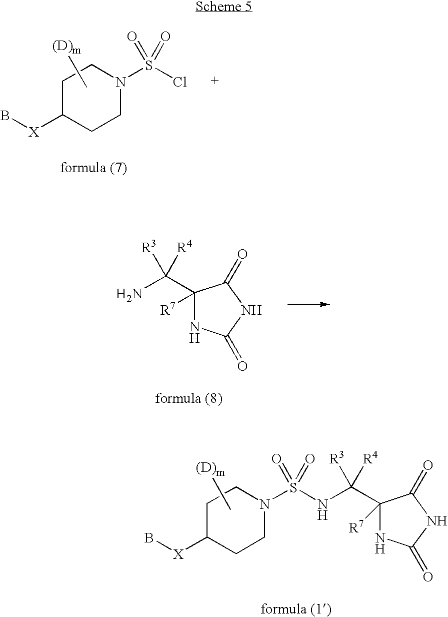

Suitable reaction conditions for such a transformation involve the addition of the sulphamoyl chloride to the amino-hydantoin in an inert solvent such as dichloromethane in the presence of a base such as triethylamine, pyridine or N,N-diisopropylethylamine at temperature of 0° C. to 50° C.

Also provided is a process for the preparation of a hydantoin of formula (8) as shown in Scheme 6:

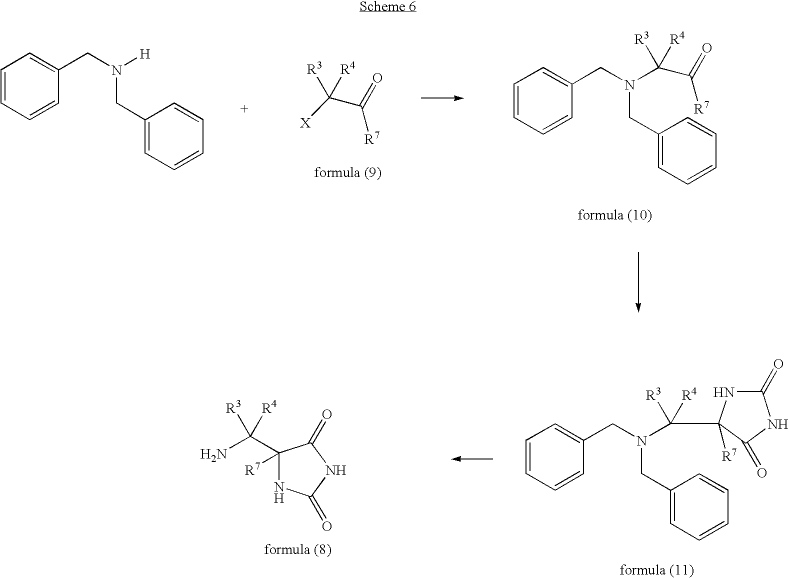

The process of Scheme 6 comprises the steps of: a) reacting dibenzylamine with a halo ketone or aldehyde (where X is halo) of formula (9) in an inert solvent such as tetrahydrofuran or dichloromethane in the presence of a base e.g triethylamine at room temperature for 24 hours to give a protected amino ketone or aldehyde of formula (10); b) reacting the ketone or aldehyde under hydantoin formation conditions to give a hydantoin of formula (11); and c) removing the benzyl protecting groups by reaction with palladium/hydrogen to yield a hydantoin of formula (8). A halo ketone or aldehyde of formula (9) is commercially available or can be prepared easily by the skilled person.

Also provided is a process for the preparation of a sulphamoyl chloride of formula (7) as shown in Scheme 7.

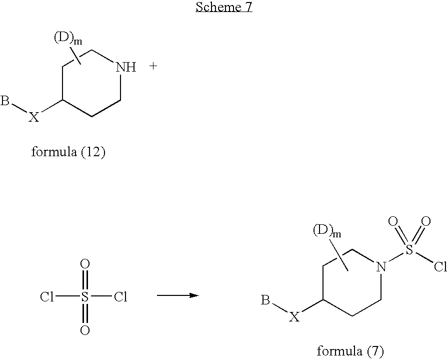

This reaction involves the treatment of a piperidine of formula (12) with sulphonyl chloride in an inert solvent in the presense of a base such as triethylamine or N,N-diisopropylethylamine. A piperidine of formula (12) is commercially available or can be easily prepared by the skilled person.

Also provided is a process for the preparation of a compound of formula (1) where W is NR¹, R¹ is hydrogen and n is 1 (indicated as a compound of formula (1″)) which process comprises reacting a sulphamoyl chloride derivative of formula (7) with an amino-hydantoin derivative of formula (13).

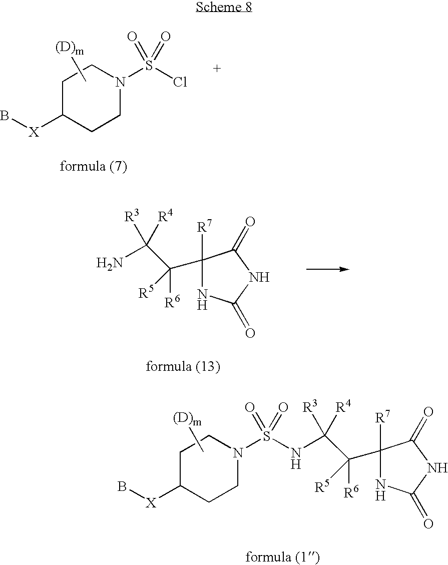

Suitable reaction conditions for such a transformation involve the addition of the sulphonyl chloride to the amino-hydantoin in an inert solvent such as dichloromethane in the presence of a base such as triethylamine, pyridine or N,N-diisopropylethylamine at temperature of 0° C. to 50° C.

Also provided is a process for the preparation of a hydantoin of formula (13), where R⁶ is hydrogen as shown in Scheme 9:

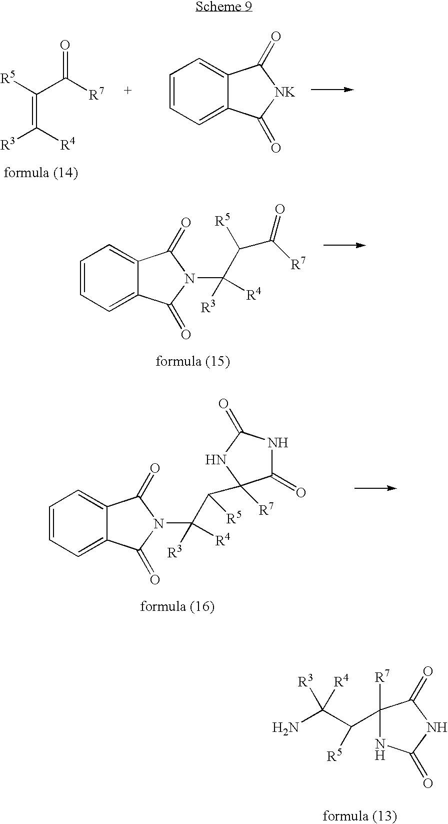

The process of Scheme 9 comprises the steps of: a) reacting an enone of formula (14) with phthalimide in the presence of sodium methoxide in an polar solvent such as dimethyl sulphoxide to give an N-substituted phthalimide of formula (15); b) forming of the hydantoin of formula (16) using e.g. ammonium carbonate and potassium cyanide in aqueous alcohols; and c) removing the phthalimide residue e.g. by reacting with HCl in acetic acid to yield a hydantoin of formula (13). An enone of formula (14) is commercially available or can be easily prepared by the skilled person.

In another aspect of the invention, there is provided a process for the preparation of compounds of formula (3) (see Scheme 2 and 4) which process is outlined in Scheme 10 and comprises; a) reacting a compound of formula (16) with a compound of formula (17) in the presence of a base to deprotonate the compound of formula (17), to yield a compound of formula (18); b) removing the protecting group (PG) from the compound of formula (18) to yield a compound of formula (19); wherein X is (CR⁹R¹⁰)ₜ-Q-(CR¹¹R¹²)ᵤ—; c) reacting the compound of formula (19) with a suitable reagent to yield a compound of formula (3);

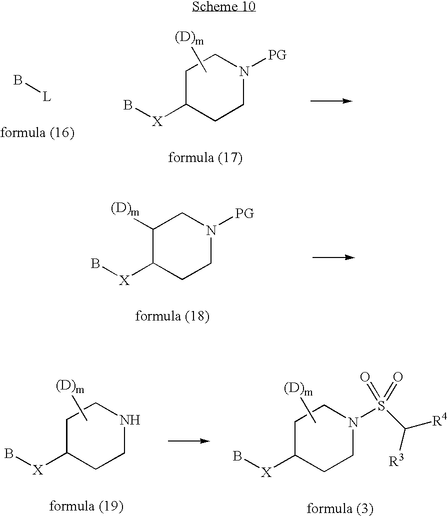

In Scheme 10: L is a suitable leaving group such as halo (chloro, bromo, iodo), mesyl, tosyl; a compound of formula (17) can be deprotonated with a base such as sodium hydride, lithium diisopropylamide, butyllithium, lithium bis(trimethylsilyl)amide and reacted with a compound of formula (16) at temperatures ranging from −78° C. to 70° C. in an aprotic solvent, e.g. tetrahydrofuran under argon; suitable protecting groups (PG) include Boc (tert-butoxycarbonyl), CBz (carbonyloxybenzyl) groups and mesyl or another alkylsulphonyl; in the case where PG is alkylsulphonyl, reaction of formula (16) and formula (17) directly produce a compound of formula (3); a compound of formula (18) can be converted to a compound of formula (19) by treatment with acid (Boc) or hydrogen/palladium (CBz); a compound of formula (19) can be converted to a compound of formula (3) by treatment with an alkylsuphonyl chloride in the presence of a base such as pyridine in a solvent such as dichloromethane.

A compound of formula (3) can also be prepared by a process as outlined in Scheme 11, which comprises; a) reacting a compound of formula (20) with a compound of formula (21), in the presence of a base to yield a compound of formula (18); b) removing the protecting group (PG) from the compound of formula (18) to yield a compound of formula (19); c) reacting the compound of formula (19) with a suitable reagent to yield a compound of formula (3); and d) oxidising Q as required.

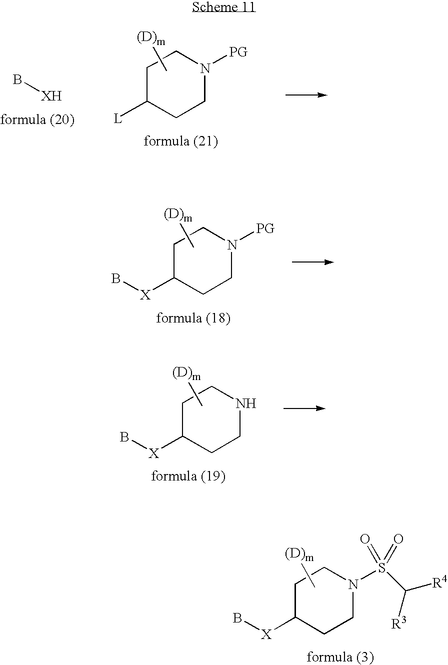

In both schemes 10 and 11: L is a suitable leaving group such as halo (chloro, bromo, iodo), hydroxy, mesyl, nosyl and tosyl; suitable bases to deprotonate compounds of formula (17) and formula (20) include bases such as caesium fluoride, sodium hydride, lithium diisopropylamide, butyllithium and lithium bis(trimethylsilyl)amide; suitable reaction conditions for step a) are temperatures ranging from −78° C. to 70° C. and in aprotic solvent, e.g. tetrahydrofuran under argon; suitable protecting groups (PG) include Boc (tell-butoxycarbonyl), CBz (carbonyloxybenzyl) groups and mesyl or another alkylsulphonyl; in the case where PG is alkylsulphonyl, reaction of formula (16) and (17) and of formula (20) and formula (21) directly produces a compound of formula (3); a compound of formula (18) can be converted to a compound formula (19) by treatment with acid (Boc) or hydrogen/palladium (CBz); a compound of formula (19) can be converted to a compound of formula (3) by treatment with an alkylsuphonyl chloride in the presence of a base such as pyridine in a solvent such as dichloromethane; and when B is aromatic, X is O and L is OH, Mitsunobu conditions can be used to form a compound of formula (18), i.e. a compound of formula (16) or formula (20) is reacted with a mixture of diethyl azodicarboxylate or diisopropylazodicarboxylate and triphenylphosphine and formula (17) or formula (21) to give a compound of formula (3). Compounds of formula (16), (17), (20) and (21) are commercially available or can be easily prepared by the skilled person.

In another aspect of the invention, a compound of formula (1) can be prepared by a process which comprises: a) reacting a sulphonyl chloride of formula (22) with a piperidine derivative of formula (19) (see scheme 10 or 11 for its preparation). 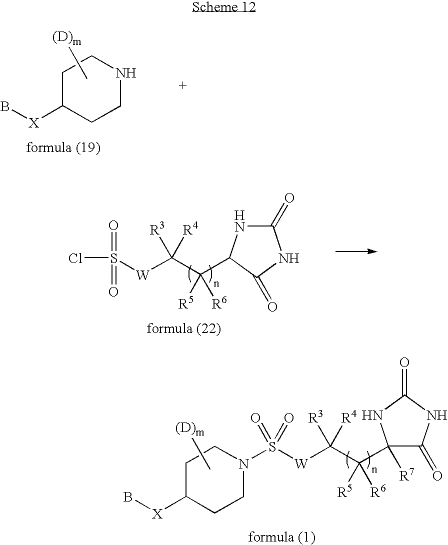
and thereafter if necessary i) converting a compound of formula (1) into another compound of formula (1); ii) removing any protecting groups; iii) forming a pharmaceutically acceptable salt or in vivo hydrolysable ester.

The sulphonyl chloride of formula (22) may be prepared as shown in scheme 13; 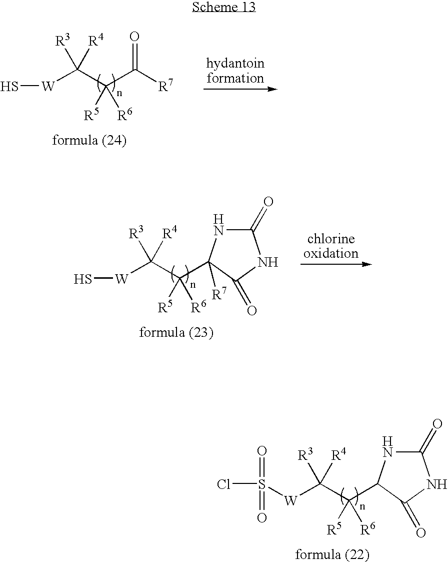
Compounds of formula (24) are readily available or can be easily made by the skilled person. Details of conditions suitable for hydantoin condition are provided herein (see scheme 1).

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses metalloproteinases inhibitory activity, and in particular TACE inhibitory activity. This property may be assessed, for example, using the procedure set out below.

Isolated Enzyme Assays

Matrix Metalloproteinase Family Including for Example MMP13.

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using 1 mM amino phenyl mercuric acid (APMA), 20 hours at 21° C.; the activated MMP13 (11.25 ng per assay) is incubated for 4-5 hours at 35° C. in assay buffer (0.1M Tris-HCl, pH 7.5 containing 0.1M NaCl, 20 mM CaCl2, 0.02 mM ZnCl and 0.05% (w/v) Brij 35 using the synthetic substrate 7-methoxycoumarin-4-yl)acetyl.Pro.Leu.Gly.Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}-Fluorescence_{background}] divided by the [Fluorescence_{minus inhibitor}-Fluorescence_{background].}

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNF-α convertase enzyme (TACE) may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate 4′,5′-Dimethoxy-fluoresceinyl Ser.Pro.Leu.Ala.Gln.Ala.Val.Arg.Ser.Ser.Ser.Arg.Cys(4-(3-succinimid-1-yl)-fluorescein)-NH₂ in assay buffer (50 mM Tris HCl, pH 7.4 containing 0.1% (w/v) Triton X-100 and 2 mM CaCl₂), at 26° C. for 4 hours. The amount of inhibition is determined as for MMP13 except λex 485 nm and λem 538 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group was removed by treating the Fmoc-peptidyl-resin with in DMF. The amino-peptidyl-resin so obtained was acylated by treatment for 1.5-2 hr at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing N,N-diisopropylethylamine, the product purified by RP-HPLC and finally isolated by freeze-drying from aqueous acetic acid. The product was characterised by MALDI-TOF MS and amino acid analysis. The compounds of this invention have bee found to be active against TACE (causing at least 50% inhibition) at 50 μm and are preferably active at 10 μm. In particular, compound 3 caused 50% inhibition at 630 nM.

Natural Substrates

The activity of the compounds of the invention as inhibitors of aggrecan degradation may be assayed using methods for example based on the disclosures of E. C. Arner et al., (1998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/Tissue Based Activity Test as an Agent to Inhibit Membrane Sheddases such as TNF Convertase

The ability of the compounds of this invention to inhibit the cellular processing of TNF-α production may be assessed in THP-1 cells using an ELISA to detect released TNF-essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47-3239-3245.

Test as an Agent to Inhibit Whole Blood TNF Sheddase Activity

The ability of the compounds of this invention to inhibit TNF-α production is assessed in a human whole blood assay where LPS is used to stimulate the release of TNF-α. 160 μl of heparinized (10 Units/ml) human blood obtained from volunteers, was added to the plate and incubated with 20 μl of test compound (duplicates), in RPMI1640 + bicarbonate, penicillin, streptomycin, glutamine and 1% DMSO, for 30 min at 37° C. in a humidified (5% CO₂/95% air) incubator, prior to addition of 20 μl LPS (E. coli. 0111:B4; final concentration 10 μg/ml). Each assay includes controls of neat blood incubated with medium alone or LPS (6 wells/plate of each). The plates are then incubated for 6 hours at 37° C. (humidified incubator), centrifuged (2000 rpm for 10 min; 4° C.), plasma harvested (50-100 μl) and stored in 96 well plates at −70° C. before subsequent analysis for TNF-α concentration by ELISA.

Test as an Agent to Inhibit in vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

In vivo Assessment

Test as an Anti-TNF Agent

The ability of the compounds of this invention as in vivo TNF-α inhibitors is assessed in the rat. Briefly, groups of female Wistar Alderley Park (AP) rats (90-100 g) are dosed with compound (5 rats) or drug vehicle (5 rats) by the appropriate route e.g. peroral (p.o.), intraperitoneal (i.p.), subcutaneous (s.c.) 1 hour prior to lipopolysaccharide (LPS) challenge (30 μg/rat i.v.). Sixty minutes following LPS challenge rats are anaesthetised and a terminal blood sample taken via the posterior vena cavae. Blood is allowed to clot at room temperature for 2 hours and serum samples obtained. These are stored at −20° C. for TNF-α ELISA and compound concentration analysis.

Data analysis by dedicated software calculates for each compound/dose:

$$Percent inhibition of TNF \text{-} α = \frac{Mean TNF \text{-} α ( Vehicle control ) - \\ Mean TNF \text{-} α ( Treated ) \times 100}{Mean TNF \text{-} α ( Vehicle control )}$$

Test as an Anti-Arthritic Agent

Activity of a compound as an anti-arthritic is tested in the collagen-induced arthritis (CIA) as defined by D. E. Trentham et al., (1977) J. Exp. Med. 146,:857. In this model acid soluble native type II collagen causes polyarthritis in rats when administered in Freunds incomplete adjuvant. Similar conditions can be used to induce arthritis in mice and primates.

Pharmaceutical Compositions

According to a further aspect of the invention there is provided a pharmaceutical composition which comprises a compound of formula (1), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository. The composition may also be in a form suitable for inhalation.

In general the above compositions may be prepared in a conventional manner using conventional excipients.

The pharmaceutical compositions of this invention will normally be administered to humans so that, for example, a daily dose of 0.5 to 75 mg/kg body weight (and preferably 0.5 to 30 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention.

Therefore in a further aspect of the present invention there is provided a compound of formula (1), or a pharmaceutically acceptable salt or in vivo hydrolysable ester thereof, as defined hereinbefore, for use in a method of treatment of a warm-blooded animal such as man by therapy. Also provided is a compound of formula (1), or a pharmaceutically acceptable salt or in vivo hydrolysable ester thereof, as defined hereinbefore, for use in a method of treating a disease condition mediated by one or more metalloproteinase enzymes and in particular a disease condition mediated by TNF-α. Further provided is a compound of formula (1), or a pharmaceutically acceptable salt or in vivo hydrolysable ester thereof, as defined hereinbefore, for use in a method of treating inflammatory diseases, autoimmune diseases, allergic/atopic diseases, transplant rejection, graft versus host disease, cardiovascular disease, reperfusion injury and malignancy in a warm-blooded animal such as man. In particular a compound of formula (1), or a pharmaceutically acceptable salt or in vivo hydrolysable ester thereof, as defined hereinbefore, is provided for use in a method of treating rheumatoid arthritis, Crohn's disease and psoriasis, and especially rheumatoid arthritis in a warm-blooded animal such as man. Also provided is a compound of formula (1), or pharmaceutically acceptable salt or in vivo hydrolysable ester thereof, for use in a method of treating a respiratory disorder such as asthma or COPD in a warm-blooded animal such as man.

According to an additional aspect of the invention there is provided a compound of formula (1), or a pharmaceutically acceptable salt or in vivo hydrolysable ester thereof, as defined hereinbefore, for use as a medicament. Also provided is a compound of formula (1), or a pharmaceutically acceptable salt or in vivo hydrolysable ester thereof, as defined hereinbefore, for use as a medicament in the treatment of a disease condition mediated by one or more metalloproteinase enzymes and in particular a disease condition mediated by TNF-α. Further provided is a compound of formula (1), or a pharmaceutically acceptable salt or in vivo hydrolysable ester thereof, as defined hereinbefore, for use as a medicament in the treatment of inflammatory diseases, autoimmune diseases, allergic/atopic diseases, transplant rejection, graft versus host disease, cardiovascular disease, reperfusion injury and malignancy. in a warm-blooded animal such as man. In particular a compound of formula (1), or a pharmaceutically acceptable salt or in vivo hydrolysable ester thereof, as defined hereinbefore, is provided for use as a medicament in the treatment of rheumatoid arthritis, Crohn's disease and psoriasis, and especially rheumatoid arthritis in a warm-blooded animal such as man. In addition, a compound of formula (1), or a pharmaceutically acceptable salt or in vivo hydrolysable ester thereof, is provided for use as a medicament in the treatment of a respiratory disorder such as asthma of COPD in a warm-blooded animal such as man.

According to this another aspect of the invention there is provided the use of a compound of formula (1), or a pharmaceutically acceptable salt or in vivo hydrolysable ester thereof, as defined hereinbefore in the manufacture of a medicament for use in the treatment of a disease condition mediated by one or more metalloproteinase enzymes and in particular a disease condition mediated by TNF-α in a warm-blooded animal such as man. Also provided is the use of a compound of formula (1), or a pharmaceutically acceptable salt or in vivo hydrolysable ester thereof, as defined hereinbefore in the manufacture of a medicament for use in the treatment of inflammatory diseases, autoimmune diseases, allergic/atopic diseases, transplant rejection, graft versus host disease, cardiovascular disease, reperfusion injury and malignancy in a warm-blooded animal such as man. In particular the use of a compound of formula (1), or a pharmaceutically acceptable salt or in vivo hydrolysable ester thereof, as defined hereinbefore, is provided in the manufacture of a medicament in the treatment of rheumatoid arthritis, Crohn's disease and psoriasis, and especially rheumatoid arthritis in a warm-blooded animal such as man. In addition, the use of a compound of formula (1), or a pharmaceutically acceptable salt or in vivo hydrolysable ester thereof, is provided in the manufacture of a medicament in the treatment of a respiratory disorder such as asthma or COPD in a warm-blooded animal such as man.

According to a further feature of this aspect of the invention there is provided a method of producing a metalloproteinase inhibitory effect in a warm-blooded animal, such as man, in need of such treatment which comprises administering to said animal an effective amount of a compound of formula (1). According to a further feature of this aspect of the invention there is provided a method of producing a TACE inhibitory effect in a warm-blooded animal, such as man, in need of such treatment which comprises administering to said animal an effective amount of a compound of formula (1). According to this further feature of this aspect of the invention there is provided a method of treating autoimmune disease, allergic/atopic diseases, transplant rejection, graft versus host disease, cardiovascular disease, reperfusion injury and malignancy in a warm-blooded animal, such as man, in need of such treatment which comprises administering to said animal an effective amount of a compound of formula (1). Also provided is a method of treating rheumatoid arthritis, Crohn's disease and psoriasis, and especially rheumatoid arthritis in a warm-blooded animal, such as man, in need of such treatment which comprises administering to said animal an effective amount of a compound of formula (1). Further provided is a method of treating a respiratory disorder such as asthma or COPD in a warm-blooded animal, such as man, in need of such treatment which comprises administering to said animal an effective amount of a compound of formula (1).

In addition to their use in therapeutic medicine, the compounds of formula (1) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

The compounds of this invention may be used in combination with other drugs and therapies used in the treatment of various immunological, inflammatory or malignant disease states which would benefit from the inhibition of TACE.

If formulated as a fixed dose such combination products employ the compounds of this invention within the dosage range described herein and the other pharmaceutically-active agent within its approved dosage range. Sequential use is contemplated when a combination formulation is inappropriate.

### EXAMPLES

The invention will now be illustrated by the following non-limiting examples in which, unless stated otherwise: (i) temperatures are given in degrees Celsius (° C.); operations were carried out at room or ambient temperature, that is, at a temperature in the range of 18-25° C.; (ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.; (iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Clamorgan, UK. Where Flashmaster II is referred to, this means a UV driven automated chromatography unit supplied by Jones; (iv) in general, the course of reactions was followed by TLC and reaction times are given for illustration only; (v) yields, when given, are for illustration only and are not necessarily those which can be obtained by diligent process development; preparations were repeated if more material was required; (vi) when given, ¹H NMR data is quoted and is in the form of delta values for major diagnostic protons, given in parts per million (ppm) relative to tetramethylsilane (TMS) as an internal standard, determined at 400 MHz using perdeuterio DMSO (CD₃SOCD₃) as the solvent unless otherwise stated; coupling constants (J) are given in Hz; (vii) chemical symbols have their usual meanings; SI units and symbols are used; (viii) solvent ratios are given in percentage by volume; (ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺; (x) LCMS (liquid chromatography mass spectrometry) characterisation was performed using a pair of Gilson 306 pumps with Gilson 233 XL sampler and Waters ZMD4000 mass spectrometer. The LC comprised water symmetry 4.6×50 column C₁₈ with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and (xi) the following abbreviations are used:

min minute(s)

h hour(s)

DIPEA N,N-diisopropylethylamine

DMSO dimethyl sulphoxide;

DMF N-dimethylformamide;

DCM dichloromethane;

NMP N-methylpyrrolidinone;

DIAD diisopropylazodicarboxylate

LHMDS or LiHMDS Lithium bis(trimethylsilyl)amide

MeOH methanol

RT room temperature

TFA trifluoroacetic acid

EtOH ethanol

EtOAc ethyl acetate.

EDTA ethylenediaminetetraacetic acid

THF tetrahydrofuran

### Example 1

### R/S-5-[({4-[(2,5-Dimethylbenzyl)oxy]piperidin-1-yl}sulphonyl)methyl]-5-methylimidazolidine-2,4-dione

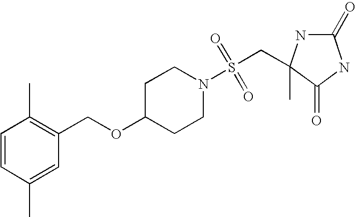

To a solution of 4-(2,5-dimethylbenzyloxy)piperidin-1-ylsulphonylpropan-2-one (prepared as described below) (210 mg, 0.62 mmol) in EtOH (14 ml) and water (6 ml) was added potassium cyanide (80 mg, 1.23 mmol) and ammonium carbonate (245 mg, 3.10 mmol). The mixture was heated at 70° C. for 5 h. Additional ammonium carbonate (1 g, 12.6 mmol) was added and the mixture stirred at RT for 17 h. The mixture was then concentrated to approximately half the volume and extracted with EtOAc (2×10 ml). The combined organic layers were partitioned with brine (10 ml), dried (MgSO₄), concentrated and purified by chromatography (10 g silica bond elute, eluent 20-100% EtOAc/Hexane) to give R/S-5-[({4-[(2,5-dimethylbenzyl)oxy]piperidin-1-yl}sulphonyl)methyl]-5-methylimidazolidine-2,4-dione as a white solid (30 mg, 0.07 mmol); NMR 1.3 (s, 3H), 1.6 (m, 2H), 1.9 (m, 2H), 2.2 (s, 3H), 2.25 (s, 3H), 3.0 (m, 2H), 3.3 (m, 3H), 3.5 (d, 1H), 3.6 (m, 1H), 4.5 (s, 2H), 7.0 (dd, 1H), 7.05 (dd, 1H), 7.1 (d, 1H), 8.0 (s, 1H), 10.7 (s, 1H); MS (−ve) 408.

The starting material, 4-(2,5-dimethylbenzyloxy)piperidin-1-ylsulphonylpropan-2-one, was prepared as described below: i) To a solution of tert-butyl 4-hydroxypiperidin-1-ylcarboxylate (4 g, 19.9 mmol) in DMF (100 ml) at RT was added sodium hydride (796 mg, 60% dispersion in oil, 19.9 mmol). After 1 h 2,5-dimethylbenzyl chloride (2.94 ml, 19.9 mmol) was added dropwise. After 16 h water was added (5 ml) and DMF was removed in vacuo. The mixture was partitioned between water (100 ml) and DCM (3×200ml) and the combined organic layers were dried (MgSO₄), concentrated and purified by chromatography (MPLC, eluting with 0→20% EtOAc/DCM) to give tert-butyl 4-(2,5-dimethylbenzyloxy)piperidin-1-ylcarboxylate as a green oil (4.15 g, 13 mmol); NMR 1.4 (m, 11H), 1.8 (m, 2H), 2.2 (d, 6H), 3.0 (m, 2H), 3.6 (m, 3H), 4.4 (s, 2H), 7.0 (m, 2H), 7.1 (s, 1H); MS: 320. ii) To a solution of tert-butyl 4-(2,5-dimethylbenzyloxy)piperidin-1-ylcarboxylate (4.1 g, 12.85 mmol) in DCM (30 ml) was added TFA (3 ml) and the mixture stirred overnight at RT. TFA (3 ml) was added and the mixture stirred at 40° C. After 1 h the mixture was concentrated and the residue azeotroped with toluene to give 4-(2,5-dimethylbenzyloxy)piperidine.TFA salt as a colourless oil (5.52 g, 12.85 mmol plus a small amount of toluene); NMR 1.7 (m, 2H), 2.0 (m, 2H), 2.2 (s, 3H), 2.25 (s, 3H), 3.0 (m, 2H), 3.2 (m, 2H), 3.65 (m, 1H), 4.45 (s, 2H), 7.0 (m, 2H) and 7.1 (s, 1H); MS: 220. iii) To a solution of 4-(2,5-dimethylbenzyloxy)piperidine.TFA salt (5.51 g, 12.85 mmol plus a small amount of toluene) in DCM (90 ml) at 0° C. was added triethylamine (8.59 ml, 61.6 mmol) followed by methanesulphonyl chloride (1.05 ml, 13.6 mmol) added dropwise over 5 min. The reaction mixture was allowed to warm to RT. After 63 h the mixture was diluted with DCM (90 ml), washed with water (50 ml), brine (50 ml), dried (MgSO₄) and concentrated to give a light brown oil. The oil was triturated with EtOH (20 ml), filtered and washed with cold EtOH and concentrated to give 4-(2,5-dimethylbenzyloxy)piperidinylsulphonylmethane as a white solid (2.63 g, 8.0 mmol); NMR 1.6 (m, 2H), 1.9 (m, 2H), 2.2 (s, 3H), 2.25 (s, 3H), 2.85 (s, 3H), 3.0 (m, 2H), 3.55 (m, 1H), 4.45 (s, 2H), 7.0 (m, 2H) and 7.1 (s, 1H); MS: 298. iv) To a stirred solution of 4-(2,5-dimethylbenzyloxy)piperidin-1-ylsulphonylmethane (500 mg, 1.68 mmol) in THF (5 ml) at 0° C., was added LHMDS (3.6 ml, 3.6 mmol). After 10 min acetyl chloride (0.14 ml, 1.96 mmol) was added. After 2 h saturated ammonium chloride (5 ml) was added, the reaction warmed to RT and the product extracted with EtOAc (2×10 ml). The combined organic layers were partitioned with brine (10 ml), dried (MgSO₄), concentrated and purified by chromatography (10 g silica bond elute, eluent 0-50% EtOAc/hexane) to give 4-(2,5-dimethylbenzyloxy)piperidin-1-ylsulphonylpropan-2-one as an oily residue (210 mg, 0.62 mmol); MS (−ve) 338.

### Example 2

### R/S-5-[({4-(2-Methylquinolin-4-ylmethoxy)piperidin-1-yl}sulphonyl)methyl]-5-methylimidazolidine-2,4-dione

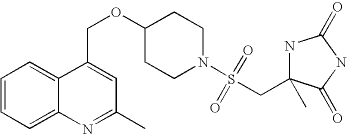

To a solution of 4-(2-methylquinolin-4-ylmethoxy)piperidin-1-ylsulphonylpropan-2-one (prepared as described below) (206 mg, 0.547 mmol) in EtOH (5 ml) and water (5 ml) was added potassium cyanide (69 mg, 1.09 mmol) and ammonium carbonate (875 mg, 3.28 mmol). The mixture was heated at 65° C. for 2 h. The mixture was then concentrated to approximately half the volume and extracted with EtOAc (3×10 ml). The combined organic layers were partitioned with brine (10 ml), dried (MgSO₄) and concentrated to give a yellow solid. This was recrystallised from hot EtOAc/iso-hexane to give R/S-5-[({4-(2-methylquinolin-4-ylmethoxy)piperidin-1-yl}sulphonyl)methyl]-5-methylimidazolidine-2,4-dione as a white solid (215 mg, 0.482 mmol); NMR 1.3 (s, 3H), 1.65 (m, 2H), 2.0 (m, 2H), 2.7 (s, 3H), 3.05 (m, 2H), 3.7 (m, 1H), 5.0 (s, 2H), 7.45 (s, 1H), 7.55 (m, 1H), 7.7 (m, 1H), 7.9 (m, 1H), 8.1 (m, 2H); MS (−ve) 445.

The starting material 4-(2-methyl-quinolin-4-yl methoxy)piperidinylsulphonylpropan-2-one, was prepared as described below: i) To a stirred suspension of 2-methylquinolin-4-ylcarboxylic acid (4 g, 21.4 mmol) in THF (100 ml) at RT was added lithium aluminium hydride (21.4 ml, 1.0M solution in THF, 21.4 mmol) dropwise over 20 min. After 16 h water (4 ml) was added cautiously followed by 2N NaOH (4 ml) and water (12 ml). The resulting gelatinous precipitate was filtered off and washed with THF. DCM (200 ml) was added to the filtrate and partitioned with saturated NaHCO₃ (2×75 ml). The organic layer was dried (MgSO₄), concentrated, triturated with DCM and filtered to give 2-methylquinolin-4-ylmethanol as a white powder (858 mg, 5 mmol). The mother liquours were purified by chromatography (20 g silica bond elute, eluent 0→5% EtOH/DCM) to give a further 610 mg of product (3.5 mmol); NMR 2.6 (s, 3H), 5.0 (d, 2H), 5.5 (t, 1H), 7.4 (s, 1H), 7.5 (t, 1H), 7.7 (t, 1H), 7.9 (m, 2H); MS: 174. ii) To a suspension of 2-methylquinolin-4-ylmethanol (100 mg, 0.58 mmol) in DCM (5 ml) at RT was added triethylamine (0.24 ml, 1.74 mmol). The reaction mixture was then cooled to 0° C. and methanesulphonyl chloride (0.05 ml, 0.64 mmol) was added dropwise. After 10 min the reaction mixture was concentrated, EtOAc (20 ml) was added and the organic layer partitioned with brine (10 ml), dried (MgSO₄), concentrated and purified by chromatography (10 g silica bond elute, eluent 5% MeOH/DCM) to give 2-methylquinolin-4-ylmethoxysulphonylmethane (110 mg, 0.44 mmol); NMR 2.7 (s, 3H), 3.35 (s, 3H), 5.75 (s, 2H), 7.5 (s, 1H), 7.6 (t, 1H), 7.75 (t, 1H), 8.0 (m, 2H); MS: 252. iii) To a solution of tert-butyl 4-hydroxypiperidin-1-ylcarboxylate (1.75 g, 8.73 mmol) in DMF (20 ml) at 0° C. was added sodium hydride (419 mg, 60% dispersion in oil, 10.5 mmol). After 10 min a solution of 2-methylquinolin-4-ylmethoxysulphonylmethane (2.19 g, 8.73 mmol) in DMF (10 ml) was added dropwise over 5 min at 0° C. After 5 h the mixture was concentrated and the residue taken up in EtOAc (150 ml). The organic layer was washed with brine (50 ml), dried (Na₂S₂O₄), concentrated and purified by chromatography (MPLC, eluting with 75% EtOAc/hexane) to give tert-butyl 4-(2-methylquinolin-4-ylmethoxy)piperidin-1-ylcarboxylate (1.46 g, 4.1 mmol); MS: 357. iv) To a solution of tert-butyl 4-(2-methylquinolin-4-ylmethoxy)piperidin-1-ylcarboxylate (1.45 g, 4.1 mmol) in DCM (10 ml) at RT was added TFA (3 ml). After 15 h the mixture was concentrated and azeotroped with toluene (x2) to give 4-(2-methylquinolin-4-ylmethoxy)piperidine.di TFA salt (1.97 g, 4.1 mmol); MS: 257. v) To a solution of 4-(2-methylquinolin-4-ylmethoxy)piperidine.di TFA salt (2.49 g, 5.2 mmol) in DCM (40 ml) at 0° C. was added triethylamine (4.3 ml, 31 mmol) followed by methanesulphonyl chloride (0.8 ml, 10.3 mmol) added dropwise over 1 min. The reaction mixture was allowed to warm to RT. After 15 h the mixture was diluted with DCM (60 ml), washed with water (30 ml), brine (25 ml), concentrated and purified by chromatography (MPLC, eluting with 100% EtOAc) to give 4-(2-methylquinolin-4-ylmethoxy)piperidin-1-ylsulphonylmethane (600 mg, 1.8 mmol) as a pale yellow solid; NMR 1.6 (m, 2H), 2.0 (m, 2H), 2.65 (s, 3H), 2.85 (s, 3H), 3.0 (m, 2H), 3.3 (m, 2H), 3.7 (m, 1H), 5.0 (s, 2H), 7.4 (s, 1H), 7.5 (t, 1H), 7.7 (t, 1H), 7.9 (d, 1H), 8.0 (d, 1H); MS: 335. vi) To a stirred solution of 4-(2-methylquinolin-4-ylmethoxy)piperidin-1-ylsulphonylmethane (400 mg, 1.06 mmol) in dry THF (10 ml) at approximately −16° C., was added LHMDS (2.63 ml, 2.34 mmol). After 30 min EtOAc (0.1 ml, 1.06 mmol) was added and the reaction warmed to RT. After 2 h saturated ammonium chloride (10 ml) was added, the reaction warmed to RT and the product extracted with EtOAc (3×20 ml). The combined organic layers were partitioned with brine (10 ml), dried (MgSO₄) and concentrated to give the crude product (0.36 g) as a yellow oil. This was titurated with iso-hexane to give 4-(2-methylquinolin-4-yl methoxy)piperidin-1-ylsulphonylpropan-2-one as a white solid (206 mg, 0.547 mmol); NMR 1.65 (m, 2H), 1.9 (m, 2H), 2.2 (s, 2H), 2.6 (s, 3H), 3.0 (m, 2H), 3.35 (m, 2H), 3.6 (m, 1H), 4.9 (s, 2H), 7.4 (s, 1H), 7.5 (t, 1H), 7.6 (t, 1H), 7.85 (d, 1H), 8.0 (d, 1H); MS (+ve) 377.

### Example 3

### 5-[2-({4-[(2-Methylquinolin-4-yl)methoxy]piperidin-1-yl}sulphonyl)ethyl]imidazolidine-2,4-dione

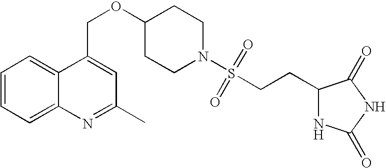

To a stirred solution of 2-methyl-4-[(piperidin-4-yloxy)methyl]quinoline. di TFA salt (example 2 step iv)) (200 mg, 0.78 mmol) in DCM (20 ml) under argon, was added DIPEA (0.4 ml, 2.35 mmol) followed by 2-(2,5-dioxo-4-imidazolidinyl)-1-ethanesulphonyl chloride (see below) (305 mg, 1.56 mmol). This was stirred for 2 h. Water (ca. 20 ml) was added and the resultant emulsion was filtered through celite. The organic phase was dried (MgSO₄) and evaporated, and the residue was purified via chromatography (silica, 1-5% MeOH/EtOAc) to give 5-[2-({4-[(2-methylquinolin-4-yl)methoxy]piperidin-1-yl}sulphonyl)ethyl]imidazolidine-2,4-dione(175 mg, 0.39 mmol) as a white solid; NMR 1.7 (m, 2H), 1.9 (m, 3H), 2.1 (m, 1H), 2.7 (s, 3H), 3.2 (m, 4H), 3.5 (m, 2H), 3.8 (m, 3H), 4.2 (m, 1H), 5.05 (s, 2H), 7.4 (s, 1H), 7.5 (t, 1H), 7.6 (s, 1H), 7.7 (t, 1H), 7.9 (d, 1H), 8.1 (d, 1H); MS 447 (MH+).

The starting material 2-(2,5-dioxo-4-imidazolidinyl)-1-ethanesulphonyl chloride was prepared as follows: i) Commercially available RS homocystine (0.18 mol) was suspended in water (25 ml). Potassium cyanate (1.5 g, 0.2 mol) was added and the mixture was stirred at 100° C. for 45 min. After partial cooling, 10% HCl (10 ml) was added and the mixture stirred at 100° C. for 50 min. The mixture was placed in the fridge overnight, and the resultant crystals were filtered, washed successively with water and dried in vacuo to afford 5-(2-{[2-(2,5-dioxo-4-imidazolidinyl)ethyl]disulphanyl}ethyl)-2,4-imidazolidinedione; LCMS (APCI) m/z 319.1 (MH+). ii) Through a suspension of 5-(2-{[2-(2,5-dioxo-4-imidazolidinyl)ethyl]disulphanyl}ethyl)-2,4-imidazolidinedione (6.9 mol) in a mixture of AcOH (25 ml) and water (2 ml) stirred vigorously at 0° C., was bubbled chlorine gas for 15 min (until all precipitate dissolved) at a maximum temperature of 5° C. The mixture was further stirred for 15 min, evaporated to a small volume in vacuo (maximum temperature 30° C.), dissolved in DCM (50 ml), shaken carefully with saturated NaHCO₃ (ca 25 ml) and then 10% sodium thiosulphate, dried, evaporated and crystallised from THF-hexane (Lora-Tamayo, M. et al, 1968, An. Quim., 64(6):591-606) to afford 2-(2,5-dioxo-4-imidazolidinyl)-1-ethanesulphonyl chloride; NMR 2.55 (m, 1.1H), 2.65 (m, 1.8H), 2.70 (m, 1H), 4.55 (m, 1H).

### Example 4

### 5-{2-[(4-{[(2-Methylquinolin-4-yl)oxy]methyl}piperidin-1-yl)sulphonyl]ethyl}imidazolidine-2,4-dione

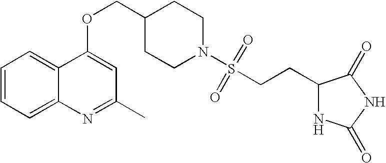

To a solution of 2-methyl-4-(piperidin-4-ylmethoxy)quinoline (hydrochloric acid salt) (50 mg, 0.17 mmol) in DMF (5 ml) was added DIPEA (0.2 ml, 1.02 mmol) followed by 2-(2,5-dioxo-4-imidazolidinyl)-1-ethanesulphonyl chloride (see example 3) (76 mg, 0.34 mmol). The reaction mixture was stirred for 3 h and then partitioned between EtOAc and water. The organic phase was dried and evaporated to give 5-{2-[(4-{[(2-methylquinolin-4-yl)oxy]methyl}piperidin-1-yl)sulphonyl]ethyl}imidazolidine-2,4-dione (25 mg, 0.056 mmol); NMR 1.4 (m, 2H), 1.9 (m, 3H), 2.15 (m, 2H), 2.6 (s, 3H), 3.2 (m, 2H), 3.7 (m, 2H), 4.2 (m, 3H), 6.9 (s, 1H), 7.45 (m, 1H), 7.7 (m, 1H), 7.8 (d, 1H), 8.1 (d, 1H), 10.4 (s, 1H); MS 447.

The starting material 2-methyl-4-(piperidin-4-ylmethoxy)quinoline (hydrochloric acid salt) was prepared as follows: i) Tert-butyl 4-(hydroxymethyl)piperidin-1-ylcarboxylate (3.0 g) was dissolved in DMF (30 ml) with stirring. Sodium hydride (60% in mineral oil, 558 mg) was then added and the mixture stirred at 80° C., under argon, for 30 min. A solution of 4-chloroquinaldine (2.5 g) in DMF (20 ml) was added, followed by potassium fluoride (100 mg) and the mixture stirred at 80° C. for 5 h. The mixture was concentrated in vacuo and the residue partitioned between EtOAc (100 ml) and water (100 ml). The aqueous phase was extracted with EtOAc (100 ml) and the combined organic layers were washed with brine (100 ml), dried (MgSO₄), concentrated in vacuo and purified on a bondelut cartridge, eluting with a gradient of 10-75% EtOAc/iso-hexane. The compound obtained was purified on a second identical cartridge, eluting with a gradient of 0-40% EtOAc/iso-hexane to give tert-butyl 4-{[(2-methylquinolin-4-yl)oxy]methyl}piperidin-1-ylcarboxylate as a white solid (2.27 g); NMR 1.25 (m, 2H), 1.40 (s, 9H), 1.82 (m, 2H), 2.10 (m, 1H), 2.55 (s, 3H), 2.75 (m, 2H), 4.00 (m, 2H), 4.10 (d, 2H), 6.92 (s, 2H), 7.45 (m, 1H), 7.65 (t, 1H), 7.82 (d, 1H), 8.05 (d, 1H); MS 357 (MH⁺). ii) Tert-butyl 4-{[(2-methylquinolin-4-yl)oxy]methyl}piperidin-1-ylcarboxylate (2.27 g) was stirred in a mixture of concentrated hydrochloric acid (12.5 ml) and 1,4-dioxane (25 ml) for 16 h. The mixture was then concentrated in vacuo, azeotroped with toluene (3×30 ml) and dried in vacuo to give 2-methyl-4-(piperidin-4-ylmethoxy)quinoline (hydrochloric acid salt) as an off-white solid (2.04 g); NMR 1.75 (m, 2H), 2.00 (m, 2H), 2.30 (m, 1H), 2.90 (s, 3H), 2.95 (m, 2H), 3.35 (m, 2H), 4.40 (m, 2H), 7.50 (s, 1H), 7.80 (t, 1H), 8.10 (t, 1H), 8.30 (d, 1H), 8.40 (d, 1H), 9.15 (m, 2H); MS 257 (MH⁺).

### Example 5

### 5-Methyl-5-{[(4-{[(2-methylquinolin-4-yl)oxy]methyl}piperidin-1-yl)sulphonyl]methyl}imidazolidine-2,4-dione

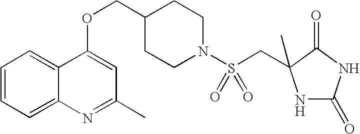

An analogous method to that described in example 4 was used except that 2-(2,5-dioxo-4-imidazolidinyl)-1-ethanesulphonyl chloride was replaced with [4-methyl-2,5-dioxoimidazolidin-4-yl]methanesulphonyl chloride. 5-methyl-5-{[(4-{[(2-methylquinolin-4-yl)oxy]methyl}piperidin-1-yl)sulphonyl]methyl}imidazolidine-2,4-dione was obtained as a white solid (53 mg); NMR 1.32 (s, 3H), 1.45 (m, 2H), 1.95 (m, 2H), 2.12 (s, 1H), 2.85 (m, 5H), 3.35 (m, 1H), 3.50 (m, 1H), 3.60 (t, 2H), 4.40 (d, 2H), 7.52 (s, 1H), 7.83 (m, 1H), 8.00 (s, 1H), 8.08 (m, 2H), 8.35 (m, 1H), 10.70 (s, 1H); MS 447 (ME⁺).

The starting material [4-methyl-2,5-dioxoimidazolidin-4-yl]methanesulphonyl chloride was prepared as follows: i) To a steel vessel charged with EtOH (315 ml) and water (135 ml) was added benzylthioacetone (31.7 g, 0.175 mol), potassium cyanide (22.9 g, 0.351 mol) and ammonium carbonate (84.5 g, 0.879 mol). The closed reaction vessel was kept at 90° C. under vigorous stirring for 3 h. The reaction vessel was then cooled with ice-water (30 min), the resultant yellowish slurry evaporated to dryness and the solid residue partitioned between water (400 ml) and EtOAc (700 ml) and separated. The aqueous phase was extracted with EtOAc (300 ml). The combined organic phases were washed with saturated brine (150 ml), dried (Na₂SO₄), filtered and evaporated to dryness. (Crystallisation was assisted by the addition of DCM (300 ml) to the oil). Evaporation gave 5-methyl-5-{[(phenylmethyl)thio]methyl}imidazolidine-2,4-dione as a slightly yellowish powder (43.8 g, 90%); NMR 1.29 (3H, s), 3.76 (2H, s), 2.72, 2.62 (1H each, ABq, J=14.0 Hz), 7.35-7.20 (5H, m), 8.00 (1H, s), 10.74 (1H, s); (MH+) m/z 251.1. ii) 5-Methyl-5-{[(phenylmethyl)thio]methyl}imidazolidine-2,4-dione (42.6 g; 0.17 mol) was dissolved in a mixture of AcOH (450 ml) and water (50 ml). The mixture was immersed in an ice/water bath and chlorine gas was bubbled through the solution, such that the temperature was maintained below 15° C. After 25 min the solution became yellow-green in colour and a sample was withdrawn for LCMS and HPLC analysis. It showed that the starting material had been consumed. The yellow clear solution was stirred for 30 min and an opaque solution/slurry was formed. The solvent was removed in vacuo at 37° C., the resultant yellowish solid was suspended in toluene (400 ml) and solvent removed in vacuo. This was repeated. The crude product was then suspended in iso-hexane (400 ml) and warmed to 40° C. while stirring. The slurry was then allowed to cool to RT before the insoluble product was removed by filtration, washed with iso-hexane (6×100 ml) and dried under reduced pressure at 50° C. overnight. This gave [4-methyl-2,5-dioxoimidazolidin-4-yl]methanesulphonyl chloride as a slightly yellow powder (36.9 g, 95%); Purity by HPLC=99%, NMR supported purity; NMR (THF-d₈) 9.91 (1H, bs), 7.57 (1H, s), 4.53, 4.44 (1H each, ABq, J=14.6 Hz), 1.52 (s, 3H, CH₃); ¹³C NMR (THF-d₈) δ 174.96, 155.86, 70.96, 61.04, 23.66.

### Example 6

### 5-Ethyl-5-[({4-[(2-methylquinolin-4-yl)methoxy]piperidin-1-yl}sulphonyl)methyl]imidazolidine-2,4-dione

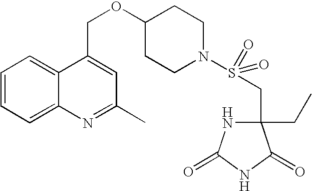

2-Methyl-4-[(piperidin-4-yloxy)methyl]quinoline.di TFA salt (example 2 step iv)) (100 mg, 0.39 mmol) was dissolved in DMF (5 ml) under argon. DIPEA (0.2 ml, 1.18 mmol) was added, followed by [4-ethyl-2,5-dioxoimidazolidin-4-yl]methanesulphonyl chloride (145 mg, 0.59 mmol) and the mixture was stirred for 20 h. The reaction mixture was then partitioned between EtOAc and water, the organic phases separated, washed with water, dried and evaporated, and the residue triturated with ether to give 5-ethyl-5-[({4-[(2-methylquinolin-4-yl)methoxy]piperidin-1-yl}sulphonyl)methyl]imidazolidine-2,4-dione as a white solid (10 mg, 0.02 mmol); NMR 0.9 (m, 3H), 1.7 (m, 4H), 2.0 (m, 2H), 2.7 (s, 3H), 3.1 (m, 2H), 3.3 (m, 3H), 3.8 (m, 1H), 5.1 (s, 2H), 7.4-7.6 (m, 3H), 7.7 (m, 1H), 7.9 (d, 1H), 8.0 (d, 1H).

The starting material [4-ethyl-2,5-dioxoimidazolidin-4-yl]methanesulphonyl chloride was prepared using an analogous method to that used in example 5 to prepare [4-methyl-2,5-dioxoimidazolidin-4-yl]methanesulphonyl chloride except that benzylthioacetone was replaced with 1-(benzylthio)butan-2-one (Tetrahedron Letters (1998), 39(20), 3189-3192.); NMR (THF-d8) 0.9 (3H, t), 1.9 (2H, m), 4.4 (1H, d), 4.5 (1H, d), 7.4 (1H, s), 9.9 (1H, s).

### Example 7

### 5-Methyl-5-[2-({4-[(2-methylquinolin-4-yl)methoxy]piperidin-1-yl}sulphonyl)ethyl]imidazolidine-2,4-dione

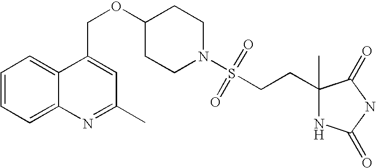

An analogous method to that described in example 6 was used except that [4-ethyl-2,5-dioxoimidazolidin-4-yl]methanesulphonyl chloride was replaced with [4-methyl-2,5-dioxoimidazolidin-4-yl]ethanesulphonyl chloride to afford 5-methyl-5-[2-({4-[(2-methylquinolin-4-yl)methoxy]piperidin-1-yl}sulphonyl)ethyl]imidazolidine-2,4-dione as an off white solid; NMR 1.29 (s, 3H), 1.70 (m, 2H), 1.97 (m, 4H), 2.89 (m, 4H), 3.08 (m, 3H), 3.44 (m, 2H), 3.78 (m, 1H), 5.23 (s, 2H), 7.81 (m, 2H), 8.01 (m, 2H), 8.16 (d, 1H), 8.37 (d, 1H), 10.73 (s, 1H); MS 461 (MH+).

The starting material [4-methyl-2,5-dioxoimidazolidin-4-yl]ethanesulphonyl chloride was prepared by an analogous method to that described in example 5 to prepare [4-methyl-2,5-dioxoimidazolidin-4-yl]methanesulphonyl chloride except that benzylthioacetone was replaced with 1-(benzylthio)butan-3-one (Angewandte Chemie, International Edition (2000), 39(23), 4316-4319); NMR (THF-d8) 1.4 (s, 3H), 2.25 (m, 1H), 2.35 (m, 1H), 3.85 (m, 1H), 4.0 (m, 1H), 7.1 (s, 1H), 9.8 (s, 1H).

### Example 8

### 5-Ethyl-5-[2-({4-[(2-methylquinolin-4-yl)methoxy]piperidin-1-yl}sulphonyl)ethyl]imidazolidine-2,4-dione

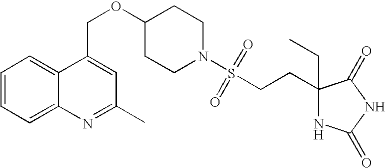

An analogous method to that described in example 6 was used except that [4-ethyl-2,5-dioxoimidazolidin-4-yl]methanesulphonyl chloride was replaced with [4-ethyl-2,5-dioxoimidazolidin-4-yl]ethanesulphonyl chloride to afford 5-ethyl-5-[2-({4-[(2-methylquinolin-4-yl)methoxy]piperidin-1-yl}sulphonyl)ethyl]imidazolidine-2,4-dione as an off white solid; NMR 0.77 (t, 3H), 1.57-1.77 (m, 4H), 1.88-2.06 (m, 4H), 2.86 (m, 4H), 3.10 (m, 3H), 3.44 (m, 2H), 3.77 (m, 1H), 5.21 (s, 2H), 7.79 (m, 2H), 7.90 (s, 1H), 8.00 (t, 1H), 8.12 (d, 1H), 8.23 (d, 1H), 10.73 (s, 1H), MS 475 (MH+).

The starting material [4-ethyl-2,5-dioxoimidazolidin-4-yl]ethanesulphonyl chloride was prepared by an analogous method to that described in example 5 to prepare [4-methyl-2,5-dioxoimidazolidin-4-yl]methanesulphonyl chloride except that benzylthioacetone was replaced with 1-(benzylthio)pentan-3-one (Chemical & Pharmaceutical Bulletin (1997), 5(5), 778-785.); NMR (THF-d8) 0.9 (t, 3H), 1.7 (m, 1H), 1.9 (m, 1H), 2.2 (m, 1H), 2.35 (m, 1H), 3.9 (m, 1H), 4.0 (m, 1H), 7.1 (s, 1H), 9.8 (s, 1H).

### Example 9

### (5S)-5-Methyl-5-{4-[(2-methylquinolin-4-yl)methoxymethyl]piperidylsulphonylmethyl}-2,4-dioxoimidazolidine

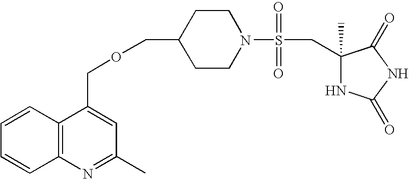

An analogous method to that described in example 6 was used except that 2-methyl-4-[(piperidin-4-yloxy)methyl]quinoline.diTFA salt was replaced with 4-[(2-methylquinolin-4-yl)methoxymethyl]piperidine and [4-ethyl-2,5-dioxoimidazolidin-4-yl]methanesulphonyl chloride replaced with (4S)-(4-methyl-2,5-dioxoimidazolidin-4-yl)methanesulphonyl chloride to afford (5S)-5-methyl-5-{4-[(2-methylquinolin-4-yl)methoxymethyl]piperidinylsulphonylmethyl}-2,4-dioxoimidazolidine as an off white solid; NMR 1.1 (m, 2H), 1.2 (m, 3H), 1.7-1.8 (m, 3H), 2.6 (s, 3H), 2.7-2.8 (m, 2H), 3.3 (m, partly obscured by H₂O, 2H), 3.3-3.5 (m, 4H), 4.9 (s, 2H), 7.4 (s, 1H), 7.5 (t, 1H), 7.7 (t, 1H), 7.9-8.1 (m, 3H), 10.7 (s, 1H); MS 461 (MH+).

The starting material 4-[(2-methylquinolin-4-yl)methoxymethyl]piperidine was prepared as follows: i) To a stirred solution of 2-methyl4-hydroxymethylquinoline (2.22 g) in DMF (40 ml) was added a 60% suspension of sodium hydride in mineral oil (620 mg). After 15 min, tert-butyl 4-({[(4-methylphenyl)sulphonyl]oxy}methyl)piperidin-1-ylcarboxylate (4.7 g) (Preparation of quinazolinyl ureas, thioureas and guanidines for use in the prevention or treatment of T cell mediated diseases or medical conditions; Crawley, McKerrecher, Poyser, Hennequin and Lambert (Astrazeneca UK Limited, UK; Zeneca Pharma S.A.) WO 0104102 169 pp) was added and the mixture stirred at 20° C. for 18 h. The mixture was quenched carefully with water (100 ml) and extracted repeatedly with EtOAc. The combined EtOAc extracts were washed with water, brine, dried and evaporated to an oil. This was chromatographed on silica in EtOAc-isohexane mixtures affording tert-butyl 4-{[(2-methylquinolin-4-yl)methoxy]methyl}piperidin-1-ylcarboxylate (1.2 g) as an oil; NMR 1.1-1.3 (m, 2H), 1.35 (s, 9H), 1.7-1.9 (m, 3H), 2.6-2.8 (s, m, 5H), 3.45 (d, 2H), 4.0-4.2 (m, 2H), 4.9 (s, 2H), 7.3 (s, 1H), 7.45 (t, 1H), 7.65 (t, 1H), 7.8 (d, 1H), 8.05 (d, 1H); MS 371.2 (MH+). ii) 4M Dioxan-HCl (40 ml) was added to a solution of tert-butyl 4-{[(2-methylquinolin-4-yl)methoxy]methyl}piperidin-1-ylcarboxylate (1.0 g) in dioxan (5 ml). MeOH (4 ml) was added and the mixture stirred at 20° C. for 2 h. The solvents were evaporated, the residue dissolved in DCM (20 ml), washed with water, NaHCO₃ and brine, dried and evaporated to a gum (1.0 g); NMR 1.2-1.3 (m, 2H), 1.7-1.9 (m, 3H), 2.5-2.75 (m, 6H), 3.15 (d, 2H), 3.45 (d, 2 H), 4.9 (s, 2H), 7.3 (s, 1H), 7.45 (t, 1H), 7.65 (t, 1H), 7.9 (d, 1H), 8.05 (d, 1H).

The starting material (4S)-(4-methyl-2,5-dioxoimidazolidin-4-yl)methanesulphonyl chloride was prepared as follows: i) A steel vessel was charged with EtOH (315 ml) and water (135 ml), and benzylthioacetone (31.7 g, 0.175 mol), potassium cyanide (22.9 g, 0.351 mol) and ammonium carbonate (84.5 g, 0.879 mol) were added. The closed reaction vessel was heated to 90° C. and stirred vigorously for 3 h. The reaction vessel was cooled with ice-water for 30 min, the yellowish slurry evaporated to dryness, the solid residue partitioned between water (400 ml) and EtOAc (700 ml) and then separated. The aqueous phase was extracted with EtOAc (300 ml) and the combined organic phases were washed with saturated brine (150 ml), dried (Na₂SO₄), filtered and evaporated to dryness. Crystallisation was assisted by the addition of DCm (300 ml). Evaporation gave 5-methyl-5-{[(phenylmethyl)thio]methyl}imidazolidine-2,4-dione as a slightly yellowish powder (43.8 g, 90%); LC-MS (APCI) m/z 251.1 (MH+); NMR 10.74 (1H, s), 8.00 (1H, s), 7.35-7.20 (5H, m), 3.76 (2H, s), 2.72, 2.62 (1H each, ABq, J=14.0 Hz), 1.29 (3H, s); ¹³C NMR DMSO-d6 177.30, 156.38, 138.11, 128.74, 128.24, 126.77, 62.93, 37.96, 36.39, 23.15. ii) (5S)-5-Methyl-5-{[(phenylmethyl)thio]methyl}imidazolidine-2,4-dione was prepared by chiral separation of the racemic material using a 250 mm×50 mm column on a Dynamic Axial Compression Preparative HPLC system. The stationary phase used was CHIRALPAK AD and other parameters were: MeOH (eluent), 89 ml/min (flow), RT, 220 nm (UV), 150 mg/ml (sample concentration) and 20 ml (injection volume). The retention time for this enantiomer under these conditions was 6 min. Analysis of chiral purity was made using a 250 mm×4.6 mm CHIRALPAK-AD column from Daicel with parameters: 0.5 ml/min (flow), EtOH (eluent), 220 nm (UV), RT. The retention time for this enantiomer under these conditions was 9.27 min. Chiral purity was estimated to >99% ee. LC-MS (APCI) m/z 251.1 (MH+); [α]_{D}=−30.3° (c=0.01 g/ml, MeOH, T=20° C.); NMR 10.74 (1H,s), 8.00 (1H, s), 7.35-7.20 (5H, m), 3.76 (2H, s), 2.72, 2.62 (1H each, ABq, J=14.0 Hz), 1.29 (3H, s); ¹³C NMR (DMSO-d6) 177.30, 156.28, 138.11, 128.74, 128.24, 126.77, 62.93, 37.96, 36.39, 23.15. (5R)-5-methyl-5-{[(phenylmethyl)thio]methyl}imidazolidine-2,4-dione was similarly prepared by chiral separation of the racemic material using a 250 mm×50 mm column on a Dynamic Axial Compression Preparative HPLC system. The stationary phase used was CHIRALPAK AD and other parameters were: MeOH (eluent), 89 ml/min (flow), RT, 220 nm (UV), 150 mg/ml (sample concentration), 20 ml (injection volume). The retention time for this enantiomer under these conditions was 10 min. Analysis of chiral purity was made using a 250 mm×4.6 mm CHIRALPAK-AD column from Daicel with parameters: 0.5 ml/min (flow), EtOH (eluent), 220 nm (UV), RT. The retention time for this enantiomer under these conditions was 17.81 min. Chiral purity was estimated to >99% ee. LC-MS (APCI) m/z 251.0 (MH+); [α]_{D}=+30.3+ (c=0.01 g/ml, MeOH, T=20° C.); NMR 10.74 (1H, s), 8.00 (1H, s), 7.35-7.20 (5H, m), 3.76 (2H, s), 2.72, 2.62 (1H each, ABq, J=14.0 Hz), 1.29 (3H, s); ¹³C NMR (DMSO-d₆) δ: 177.31, 156.30, 138.11, 128.74, 128.25, 126.77, 62.94, 37.97, 36.40, 23.16. iii) (5S)-5-Methyl-5-{[(phenylmethyl)thio]methyl}imidazolidine-2,4-dione (42.6 g, 0.17 mol) was dissolved in a mixture of AcOH (450 ml) and water (50 ml). The mixture was immersed in an ice/water bath and chlorine gas was bubbled through the solution, the flow of gas being adjusted to maintain the temperature at below 15° C. After 25 min the solution became yellow-green in colour and a sample was withdrawn for LC/MS and HPLC analysis. It showed that starting material had been consumed. The resultant clear yellow solution was stirred for 30 min and an opaque solution/slurry was formed. The solvent was removed on a rotary evaporator at 37° C., the resultant yellowish solid suspended in toluene (400 ml) and solvent removed on the same rotary evaporator. This was repeated once more. The crude product was then suspended in iso-hexane (400 ml) and warmed to 40° C. while stirring. The slurry was allowed to cool to RT before the insoluble product was removed by filtration, washed with iso-hexane (6×100 ml), and dried under reduced pressure at 50° C. over night. This gave [(4S)-4-methyl-2,5-dioxoimidazolidin-4-yl]methanesulphonyl chloride as a slightly yellow powder (36.9 g, 95%); purity by HPLC=99%, NMR supported that purity; [α]_{D}=−12.4° (c=0.01 g/ml, THF, T=20° C.); NMR (THB-d₈) 9.91 (1H, bs), 7.57 (1H, s), 4.53, 4.44 (1H each, ABq, J=14.6 Hz), 1.52 (s, 3H, CH₃); ¹³C NMR (TBF-d₈) 174.96, 155.86, 70.96, 61.04, 23.66. [(4R)-4-Methyl-2,5-dioxoimidazolidin-4-yl]methanesulphonyl chloride can be prepared by the same method; [α]_{D}=+12.8° (c=0.01 g/ml, THF, T=20° C.), NMR (THF-d₈) 9.91 (1H, brs), 7.57 (1H, s), 4.53, 4.44 (1H each, ABq, J=14.6 Hz), 1.52 (s, 3H, CH₃); ¹³C NMR (THF-d₈) 174.96, 155.84, 70.97, 61.04, 23.66.

### Example 10

### (5S)-5-Ethyl-5-{4-[(2-methylquinolin-4-yl)methoxymethyl]piperidylsulphonylmethyl}-2,4-dioxoimidazolidine

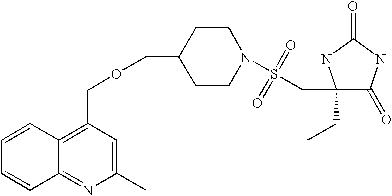

An analogous method to that described in example 6 was used except that 2-methyl-4-[(piperidin-4-yloxy)methyl]quinoline was replaced with 4-[(2-methylquinolin-4-yl)methoxymethyl]piperidine and [4-ethyl-2,5-dioxoimidazolidin-4-yl]methanesulphonyl chloride was replaced with (4S)-2-(4-ethyl-2,5-dioxoimidazolidin-4-yl)methanesulphonyl chloride to afford (5S)-5-ethyl-5-{4-[(2-methylquinolin-4-yl)methoxymethyl]piperidylsulphonylmethyl}-2,4-dioxoimidazolidine as an off white solid; NMR 0.75 (t, 3H), 1.1-1.3 (m, 2H), 1.6 (q, 2H), 1.7-1.85 (m, 3H), 2.65 (s, 3H),2.7-2.9 (m, 2H), 3.2-3.3 (m, 2H), 3.3-3.6 (m, 4H), 4.9 (s, 2H), 7.4 (s, 1H), 7.5 (t, 1H), 7.7 (t, 1H), 7.8 (m, 2H), 8.0 (d, 1H), 10.7 (s, 1H); MS 473 (MH−).

The starting material 2-methyl-4-[(piperidin-4-yloxy)methyl]quinoline was prepared as described in example 9 and (4S)-2-(4-ethyl-2,5-dioxoimidazolidin-4-yl)methanesulphonyl chloride was prepared as described in example 6. Separation of the isomers was achieved as described in example 9.

(5S)-5-Ethyl-5-{[(phenylmethyl)thio]methyl}imidazolidine-2,4-dione: the retention time for this enantiomer was 4.1 min; NMR 0.77 (t, 3H), 1.59 (m, 2H), 2.67 (q, 2H), 3.76 (s, 2H), 7.24 (m, 1H), 7.31 (m, 4H), 7.89 (s, 1H), 10.72 (s, 1H).

[(4S)-4-Ethyl-2,5-dioxoimidazolidin-4-yl]methanesulphonyl chloride; NMR (TBF-d₈) 0.96 (s, 3H), 1.90 (m, 2H); 4.49 (d, 1H), 4.59 (d, 1H), 7.54 (s, 1H), 9.97 (s, 1H).

1. A compound of formula (1) or a pharmaceutically acceptable salt thereof: 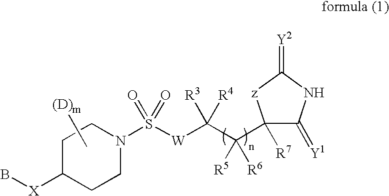
wherein:
Y¹ and Y² are independently O or S;
z is NR⁸, O or S;
n is 0 or 1;
W is NR¹, CR¹R² or a bond;
m is 0 or 1;
D is hydrogen, C₁₋₄alkyl, C₃₋₆cycloalkyl or fluoro;
X is —(CR¹²R¹³)ₜ-Q-(CR¹⁴R¹⁵)ᵤ— where t and u are independently 0 or 1 and Q is O, S, SO or SO₂;
B is a group selected from aryl, heteroaryl and heterocyclyl, where each group is optionally substituted by one or more groups independently selected from nitro, trifluoromethyl, trifluoromethoxy, halo, cyano, C₁₋₄alkyl [symbol] optionally substituted by R⁹ or C₁₋₄alkoxy or one or more halo [symbol] , C₂₋₄alkenyl [symbol] optionally substituted by halo or R⁹ [symbol] , C₂₋₄alkynyl [symbol] optionally substituted by halo or R⁹ [symbol] , C₃₋₆cycloalkyl [symbol] optionally substituted by R⁹ or one or more halo [symbol] , C₅₋₆cycloalkenyl (optionally substituted by halo or R⁹ [symbol] , aryl [symbol] optionally substituted by halo or C₁₋₄alkyl [symbol] , heteroaryl [symbol] optionally substituted by halo or C₁₋₄alkyl [symbol] , heterocyclyl [symbol] optionally substituted by C₁₋₄alkyl [symbol] , —SR¹¹, −SOR¹¹, —SO₂R¹¹, −SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy; with the provisos that:
when n is 1 and W is NR¹, CR¹R² or a bond; or when n is 0 and W is CR¹R²; then B is a group selected from aryl, heteroaryl and heterocyclyl, where each group is optionally substituted by one or more groups independently selected from nitro, trifluoromethyl, trifluoromethoxy, halo, cyano, C₁₋₄alkyl [symbol] optionally substituted by R⁹ or C₁₋₄alkoxy or one or more halo [symbol] , C₂₋₄alkenyl [symbol] optionally substituted by halo or R⁹ [symbol] , C₂₋₄alkynyl [symbol] optionally substituted by halo or R⁹ [symbol] , C₃₋₆cycloalkyl [symbol] optionally substituted by R⁹ or one or more halo [symbol] , C₅₋₆cycloalkenyl [symbol] optionally substituted by halo or R⁹ [symbol] , aryl [symbol] optionally substituted by halo or C₁₋₄alkyl [symbol] , heteroaryl [symbol] optionally substituted by halo or C₁₋₄alkyl [symbol] , heterocyclyl [symbol] optionally substituted by C₁₋₄alkyl [symbol] , —SR¹¹, —SOR¹¹, —SO₂R¹¹, —SO₂NR⁹R¹⁰ , —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy; and
when n is 0 and W is NR¹ or a bond; then B is a group selected from bicyclic aryl, bicyclic heteroaryl and bicyclic heterocyclyl, where each group is optionally substituted by one or more groups independently selected from nitro, trifluoromethyl, trifluoromethoxy, halo, cyano, C₁₋₄alkyl [symbol] optionally substituted by R⁹ or C₁₋₄alkoxy or one or more halo [symbol] , C₂₋₄alkenyl [symbol] optionally substituted by halo or R⁹ [symbol] , C₂₋₄alkynyl [symbol] optionally substituted by halo or R⁹ [symbol] , C₃₋₆cycloalkyl [symbol] optionally substituted by R⁹ or one or more halo [symbol] , C₅₋₆cycloalkenyl [symbol] optionally substituted by halo or R⁹ [symbol] , aryl [symbol] optionally substituted by halo or C₁₋₄alkyl [symbol] , heteroaryl [symbol] optionally substituted by halo or C₁₋₄alkyl [symbol] , heterocyclyl [symbol] optionally substituted by C₁₋₄alkyl [symbol] , —SR¹¹, —SOR¹¹, —SO₂R¹¹, —SO₂NR⁹R¹⁰, —NR⁹SO₂R¹¹, —NHCONR⁹R¹⁰, —OR⁹, —NR⁹R¹⁰, —CONR⁹R¹⁰ and —NR⁹COR¹⁰; or B is C₂₋₄alkenyl or C₂₋₄alkynyl, each being optionally substituted by a group selected from C₁₋₄alkyl, C₃₋₆cycloalkyl, aryl, heteroaryl and heterocyclyl which group is optionally substituted by one or more halo, nitro, cyano, trifluoromethyl, trifluoromethoxy, —CONHR⁹, —CONR⁹R¹⁰, —SO₂R¹¹, —SO₂NR⁹R¹⁰, —NR⁹SO₂R¹¹, C₁₋₄alkyl or C₁₋₄alkoxy;
R¹ and R² are independently hydrogen or a group selected from C₁₋₆alkyl, C₂₋₆alkenyl, C₂₋₆alkynyl, C₃₋₆cycloalkyl and C₅₋₆cycloalkenyl which group may be optionally substituted by halo, cyano, hydroxy or C₁₋₄alkoxy;
R³, R⁴, R⁵ and R⁶ are independently hydrogen or a group selected from C₁₋₆alkyl, C₂₋₆alkenyl, C₂₋₆alkynyl, C₃₋₆cycloalkyl, C₅₋₆cycloalkenyl, aryl, heteroaryl and heterocyclyl which group is optionally substituted by one or more substituents independently selected from halo, nitro, cyano, trifluoromethyl, trifluoromethyloxy, C₁₋₄alkyl, C₂₋₄alkenyl, C₂₋₄alkynyl, C₃₋₆cycloalkyl [symbol] optionally substituted by one or more R¹⁷ [symbol] , aryl [symbol] optionally substituted by one or more R¹⁷ [symbol] , heteroaryl [symbol] optionally substituted by one or more R¹⁷ [symbol] , heterocyclyl, —OR¹⁸, —SR¹⁹, —SOR¹⁹, —SO₂R¹⁹, —COR¹⁹, —CO₂R¹⁸, —CONR¹⁸R²⁰, —NR¹⁶COR¹⁸, —SO₂NR¹⁸R²⁰ and —NR¹⁶SO₂R¹⁹;
or R¹ and R³ together with the nitrogen or carbon atoms and carbon atom to which they are respectively attached form a saturated 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
or R³ and R⁴ together form a saturated 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
or R⁵ and R⁶ together form a saturated 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
R⁷ is hydrogen or a group selected from C₁₋₆alkyl, C₂₋₆alkenyl, C₂₋₆alkynyl, heteroalkyl, C₃₋₇cycloalkyl, aryl, heteroaryl or heterocyclyl where the group is optionally substituted by halo, C₁₋₄alkyl, C₁₋₄alkoxy, C₃₋₇cycloalkyl, heterocyclyl, aryl, heteroaryl or heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₇cycloalkyl, heterocyclyl, C₁₋₄alkoxyC₁₋₄alkyl, haloC₁₋₄alkoxyC₁₋₄alkyl, —COC₁₋₄alkyl, —OR²¹, —CO₂R²¹, —SR²⁵, —SOR²⁵, —SO₂R²⁵, —NR²¹COR²², CONR²¹R²² and —NHCONR²¹R²²;
or R³ and R⁷ together with the carbon atoms to which they are each attached and (CR⁵R⁶)ₙ form a saturated 5- to 7-membered ring optionally containing a heteroatom group selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
R⁸ is selected from hydrogen, C₁₋₆alkyl and haloC₁₋₆alkyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4 to 7-membered ring.
R¹¹ is C₁₋₆alkyl or C₃₋₆cycloalkyl;
R¹², R¹³, R¹⁴ and R¹⁵ are independently selected from hydrogen, C₁₋₆alkyl and C₃₋₆cycloalkyl;
R¹⁶ is hydrogen or C₁₋₆alkyl;
R¹⁷ is selected from halo, C₁₋₆alkyl, C₃₋₆cycloalkyl and C₁₋₆alkoxy;
R¹⁸ is hydrogen or a group selected from C₁₋₆alkyl, C₃₋₆cycloalkyl, C₅₋₇cycloalkenyl, saturated heterocyclyl, aryl, heteroaryl, arylC₁₋₄alkyl and heteroarylC₁₋₄alkyl which group is optionally substituted by one or more halo;
R¹⁹ and R²⁵ are independently a group selected from C¹⁻⁶alkyl, C₃₋₆cycloalkyl, C₅₋₇cycloalkenyl, saturated heterocyclyl, aryl, heteroaryl, arylC₁₋₄alkyl and heteroarylC₁₋₄alkyl which group is optionally substituted by one or more halo;
R²⁰ is hydrogen, C₁₋₆alkyl or C₃₋₆cycloalkyl;
or R¹⁸ and R²⁰ together with the nitrogen to which they are attached form a heterocyclic 4- to 7-membered ring;
R²¹ and R²² are independently hydrogen, C₁₋₄alkyl, haloC₁₋₄alkyl, aryl and arylC₁₋₄alkyl;
or R²¹ and R²² together with the nitrogen to which they are attached form a heterocyclic 5- to 6-membered ring.

2. A compound of formula (1) or a pharmaceutically acceptable salt thereof: 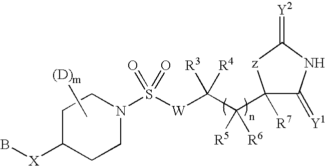
wherein:
Y¹ and Y² are independently O or S;
z is NR⁸, O or S;
n is 0;
W is NR¹;
m is 0 or 1;
D is hydrogen, C₁₋₄alkyl, C₃₋₆cycloalkyl or fluoro;
X is —(CR¹²R¹³)ₜ-Q-(CR¹⁴R¹⁵)ᵤ where t and u are independently 0 or 1 and Q is O, S, SO or SO₂;
B is a group selected from aryl, heteroaryl and heterocyclyl, where each group is optionally substituted by one or more groups independently selected from nitro, trifluoromethyl, trifluoromethoxy, halo, cyano, C₁₋₄alkyl [symbol] optionally substituted by R⁹ or C₁₋₄alkoxy or one or more halo [symbol] , C₂₋₄alkenyl [symbol] optionally substituted by halo or R⁹ [symbol] , C₂₋₄alkynyl [symbol] optionally substituted by halo or R⁹ [symbol] , C₃₋₆cycloalkyl [symbol] optionally substituted by R⁹ or one or more halo [symbol] , C₅₋₆cycloalkenyl [symbol] optionally substituted by halo or R⁹ [symbol] , aryl [symbol] optionally substituted by halo or C₁₋₄alkyl [symbol] , heteroaryl [symbol] optionally substituted by halo or C₁₋₄alkyl [symbol] , heterocyclyl [symbol] optionally substituted by C₁₋₄alkyl), —SR¹¹, —SOR¹¹, —SO₂R¹¹, —SO₂NR⁹R¹⁰, —NR⁹SO₂R¹¹, —NHCONR⁹R¹⁰, —OR⁹, —CONR⁹R¹⁰ and —NR⁹COR¹⁰;
R¹ is hydrogen or a group selected from C₁₋₆alkyl, C₂₋₆alkenyl, C₂₋₆alkynyl, C₃₋₆cycloalkyl and C₅₋₆cycloalkenyl which group may be optionally substituted by halo, cyano, hydroxy or C₁₋₄alkoxy;
R³ and R⁴ are independently hydrogen or a group selected from C₁₋₄alkyl, C₂₋₄alkenyl, C₂₋₄alkynyl, C₃₋₅cycloalkyl, pentenyl, aryl, heteroaryl and heterocyclyl which group is optionally substituted by one or more substituents independently selected from halo, nitro, cyano, trifluoromethyl, trifluoromethyloxy, C₁₋₄alkyl, C₂₋₄alkenyl, C₂₋₄alkynyl, C₃₋₆cycloalkyl [symbol] optionally substituted by one or more R¹⁷ [symbol] , aryl [symbol] optionally substituted by one or more R¹⁷ [symbol] , heteroaryl [symbol] optionally substituted by one or more R¹⁷ [symbol] , heterocyclyl, —OR¹⁸, —SR¹⁹, —SOR¹⁹, —SO₂R¹⁹, —CONR¹⁸R²⁰ and —NR¹⁶COR¹⁸;
or R¹ and R³ together with the nitrogen and carbon atoms to which they are respectively attached form a saturated 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
or R³ and R⁴ together form a carbocyclic or saturated heterocyclic 3- to 7-membered ring optionally containing 1 or 2 heteroatom groups selected from NH, O, S, SO and SO₂ where the ring is optionally substituted on carbon by C₁₋₄alkyl, fluoro or C₁₋₄alkoxy and/or on nitrogen by —COC₁₋₃alkyl, —SO₂C₁₋₃alkyl or C₁₋₄alkyl;
R⁷ is hydrogen or a group selected from C₁₋₄alkyl, heteroalkyl, C₃₋₅cycloalkyl, aryl, heteroaryl or heterocyclyl which group is optionally substituted by halo, C₁₋₄alkyl, C₁₋₄alkoxy, C₃₋₅cycloalkyl, heterocyclyl, aryl, heteroaryl or heteroalkyl; and wherein the group from which R⁷ may be selected is optionally substituted on the group and/or on its optional substituent by one or more substituents independently selected from halo, cyano, C₁₋₄alkyl, nitro, haloC₁₋₄alkyl, heteroalkyl, aryl, heteroaryl, hydroxyC₁₋₄alkyl, C₃₋₅cycloalkyl, heterocyclyl, C₁₋₄alkoxyC₁₋₄alkyl, haloC₁₋₄alkoxyC₁₋₄alkyl, —COC₁₋₄alkyl, —OR²¹, —CO₂R²¹, —SR²⁵, —SOR²⁵, —SO₂R²⁵, —CONR²¹R²² and —NHCONR²¹R²²;
or R³ and R⁷ together with the carbon atoms to which they are each attached and (CR⁵R⁶)ₙ form a saturated carbocyclic or heterocyclic 5- or 6-membered ring;
R⁸ is selected from hydrogen, C₁₋₄alkyl and haloC₁₋₄alkyl;
R⁹ and R¹⁰ are independently hydrogen, C₁₋₆alkyl or C₃₋₆cycloalkyl;
or R⁹ and R¹⁰ together with the nitrogen to which they are attached form a heterocyclic 4 to 6-membered ring.
R¹¹ is C₁₋₄alkyl or C₃₋₅cycloalkyl;
R¹², R¹³, R¹⁴ and R¹⁵ are independently selected from hydrogen, C₁₋₄alkyl and C₃₋₄cycloalkyl;
R¹⁶ is hydrogen or C₁₋₄alkyl;
R¹⁷ is selected from halo, C₁₋₄alkyl, C₃₋₅cycloalkyl and C₁₋₄alkoxy;
R¹⁸ is hydrogen or a group selected from C₁₋₄alkyl, C₃₋₅cycloalkyl, C₅₋₆cycloalkenyl, saturated heterocyclyl, aryl, heteroaryl, arylC₁₋₄alkyl and heteroarylC₁₋₄alkyl which group is optionally substituted by one or more halo;
R¹⁹ and R²⁵ are independently a group selected from C₁₋₄alkyl, C₃₋₅cycloalkyl, C₅₋₆cycloalkenyl, saturated heterocyclyl, aryl, heteroaryl, arylC₁₋₄alkyl and heteroarylC₁₋₄alkyl which group is optionally substituted by one or more halo;
R²⁰ is hydrogen, C₁₋₄alkyl or C₃₋₅cycloalkyl;
or R¹⁸ and R²⁰ together with the nitrogen to which they are attached form a heterocyclic 4- to 6-membered ring;
R²¹ and R²² are independently hydrogen, C₁₋₄alkyl, haloC₁₋₄alkyl, aryl and arylC₁₋₄alkyl;
or R²¹ and R²² together with the nitrogen to which they are attached form a heterocyclic 5- to 6-membered ring.

3. A compound according to claim 1 wherein B is phenyl, naphthyl, pyridyl, quinolinyl, isoquinolinyl, thienopyridyl, naphthyridinyl, 2,3-methylenedioxyphenyl, 3,4-methylenedioxyphenyl, thienopyrimidinyl, pyridoimidazolyl, benzimidazolyl, benzofuranyl, benzothienyl, indolyl, benzothiazolyl, benzotriazolyl, benzisoxazolyl, benzisothiazolyl, indazolyl, indolizinyl, isobenzofuranyl, quinazolinyl, imidazopyridinyl, pyrazolopyridinyl, indolinyl, tetrahydroquinolinyl, tetrahydroisoquinolinyl or isoindolinyl, where each is optionally substituted by one or more groups independently selected from nitro, trifluoromethyl, trifluoromethoxy, halo, C₁₋₄alkyl [symbol] optionally substituted by one or more halo [symbol] , C₂₋₄alkynyl, heteroaryl, —OR⁹, cyano, —NR⁹R¹⁰, —CONR⁹R¹⁰ and —NR⁹COR¹⁰; or B is vinyl or ethynyl optionally substituted by C₁₋₄alkyl.

4. A compound according to claim 1 wherein B is a group selected from bicyclic aryl, bicyclic heteroaryl and bicyclic heterocyclyl, where each group is optionally substituted by one or more groups independently selected from nitro, trifluoromethyl, trifluoromethoxy, halo, C₁₋₄alkyl [symbol] optionally substituted by one or more halo [symbol] , C₂₋₄alkynyl, heteroaryl, —OR⁹, cyano, —NR⁹R¹⁰, —CONR⁹R¹⁰ and —NR⁹COR¹⁰; or B is C₂₋₄alkenyl or C₂₋₄alkynyl optionally substituted by C₁₋₄alkyl, C₃₋₆cycloalkyl or heterocyclyl.

5. A compound according to claim 1 wherein B is 2-methylquinolin-4-yl.

6. A compound according to claim 1 wherein R⁷is hydrogen or a group selected from C₁₋₄alkyl, arylC₁₋₄alkyl, heteroarylC₁₋₄alkyl, heterocyclylC₁₋₄alkyl, aryl, heteroaryl, heterocyclyl and C₃₋₅cycloalkyl which group is optionally substituted by cyano, C₁₋₄alkyl, halo, —OR²¹, —NR²¹R²², —CO₂R²¹ and —NR²¹CO₂R²².

7. A compound according to claim 6 wherein R⁷ is hydrogen or C₁₋₄alkyl optionally substituted with halo, hydroxy or C₁₋₃alkoxy.

8. A pharmaceutical composition comprising a compound according to claim 1; and a pharmaceutically-acceptable diluent or carrier.

9. (canceled)

10. A method of treating a disease condition mediated by TNF-α comprising administering to an animal an effective amount of a compound of claim 1.

11. (canceled)

12. A method of treating autoimmune disease, allergic/atopic diseases, transplant rejection, graft versus host disease, cardiovascular disease, reperfusion injury and malignancy which comprises administering a compound according to claim 1.

13. A process for preparing a compound according to claim 1, comprising the steps of converting a ketone or aldehyde of formula (2) into a compound of formula (1); 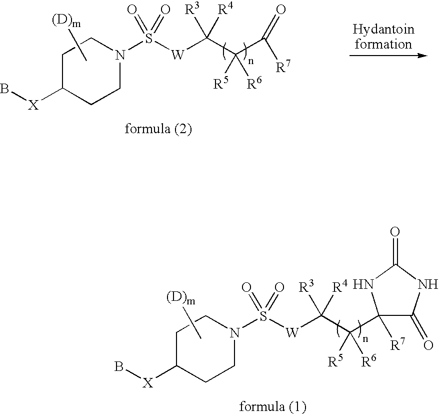
and thereafter if necessary:
i) converting a compound of formula (1) into another compound of formula (1);
ii) removing any protecting groups;
iii) forming a pharmaceutically acceptable salt or in vivo hydrolysable ester.
